(19) United States
(12) Patent Application Publication
(10) Pub. No.: US 2026/0226010 A1
(43) Pub. Date: Aug. 6, 2026

(54) HETEROCYCLIC COMPOUNDS AS PI3Ka INHIBITORS

(71) Applicant: Pikavation Therapeutics, Inc., Wilmington, DE (US)

(72) Inventors: Yu Bai, Wilmington, DE (US); Chao Qi, Wilmington, DE (US); Liangxing Wu, Wilmington, DE (US); Wenqing Yao, Wilmington, DE (US)

(21) Appl. No.: 19/152,154

(22) PCT Filed: Feb. 1, 2024
(86) PCT No.: PCT/US2024/014022

§ 371 Date: Jul. 30, 2025

Related U.S. Application Data
(60) Provisional application No. 63/442,806, filed on Feb. 2, 2023.

(51) Int. Cl. C07D 221/20; A61K 31/4747; C07D 401/12; C07D 401/14

(52) U.S. Cl. CPC C07D 221/20; A61K 31/4747; C07D 401/12; C07D 401/14

(57) ABSTRACT

The present application provides heterocyclic compounds that modulate the activity of the PI3Kα, which are useful in the treatment of various diseases, including cancer. (I)

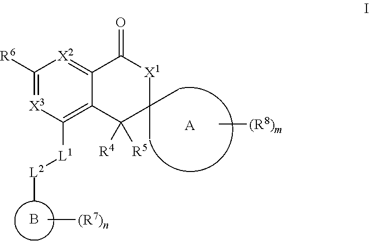

### TECHNICAL FIELD

The present disclosure provides heterocyclic compounds as well as their pharmaceutical compositions that modulate the activity of PI3Kα and are useful in the treatment of various diseases related to PI3Kα, including cancer.

### BACKGROUND

In the past few decades, signal transduction events have been studied to demonstrate critical roles in regulating almost all aspects of biological responses. Aberrant activation of the signaling pathways regulating cell survival and proliferation is commonly observed in many human cancers. The phosphoinositide 3-kinases (PI3Ks) signaling pathway is documented to be one of the highly mutated pathways in human cancers (Vogelstein et al., Science, 2013, 339(6127), 1546-1558). The PI3K signaling pathway regulates cell survival and proliferation. Increased activity of this pathway is associated with tumor progression and resistance to cancer therapies (Fusco et al., Front Oncol, 2021, 11, 644737).

PI3Ks belong to a lipid kinase family which catalyzes the phosphorylation of lipids contained in or associated with cell membranes. The PI3K family has fifteen kinases with distinct substrates, expression pattern, and modes of regulation. The class-I PI3Ks (p110α, p110β, p110δ, and p110γ) are typically activated by tyrosine receptor kinases or G-protein coupled receptors to generate PIP3, which activates downstream effectors of Akt, mTOR, or Rho GTPases (Fruman et al., Nat. Rev. Drug Discov., 2014, 13(2), 140-156) Genetic mutations in the gene coding for PI3Kα are hotspot point mutations within helical and kinase domains, such as E542K, E545K, and H1047R. These mutations have been observed to occur in many cancer types such as lung, stomach, endometrial, ovarian, bladder, breast, colon, brain, prostate, and skin cancers. Because these gain-of-function mutations in PI3Kα are associated with tumor progression, targeting this pathway may provide valuable therapeutic opportunities (Courtney et al., J. Clin. Oncol., 2010, 28 (6), 1075-1083). While multiple inhibitors of PI3Ks have been developed (for example, taselisib, alpelisib, buparlisib and others), these molecules inhibit multiple PI3K isoforms. These “pan-PI3K” inhibitors have encountered major hurdle in the clinical development due to inability to achieve the required level of target inhibition in tumors while avoiding toxicity in cancer patients (Fruman et al., Nat. Rev. Drug Discov., 2014, 13(2), 140-156). The toxicity of PI3K inhibitors is dependent on their isoform selectivity profile. Inhibition of PI3Kα is associated with hyperglycemia and rash, while inhibition of PI3Kδ or PI3Kγ is associated with diarrhea, myelosuppression, and transaminitis (Hanker et al., Cancer Discov., 2019, 9(4), 482-491). Therefore, selective inhibitors of PI3Kα may increase the therapeutic window, enabling sufficient target inhibition in the tumor while avoiding dose-limiting toxicity in cancer patients. However, given the central role of PI3Kα in regulating glucose homeostasis and other critical physiological process, current PI3Kα selective inhibitors, which are equally potent to wild-type and mutant PI3Kα, often cause hyperglycemia and/or hyperinsulinemia (Busaidy et al., J. Clin. Oncol., 2012, 30, 2919-2928). In summary, developing inhibitors with enhanced selectivity for mutant PI3Kα against wild-type PI3Kα would be able to overcome the problem of compensatory insulin production and hyperglycemia.

### SUMMARY

The present disclosure provides, inter alia, compounds of Formula I.

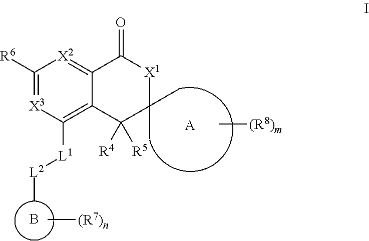

or pharmaceutically acceptable salts thereof, wherein constituent members are defined herein.

The present disclosure further provides a pharmaceutical composition comprising a compound of the disclosure, or a pharmaceutically acceptable salt thereof, and a pharmaceutically acceptable carrier or excipient.

The present disclosure further provides methods of inhibiting PI3Kα activity, comprising contacting the PI3Kα with a compound described herein, or a pharmaceutically acceptable salt thereof.

The present disclosure further provides methods of treating a disease or a disorder associated with PI3Kα in a patient by administering to the patient a therapeutically effective amount of a compound of the disclosure, or a pharmaceutically acceptable salt thereof.

The present disclosure further provides a compound described herein, or a pharmaceutically acceptable salt thereof, for use in any of the methods described herein.

The present disclosure further provides use of a compound described herein, or a pharmaceutically acceptable salt thereof, for the preparation of a medicament for use in any of the methods described herein.

### DETAILED DESCRIPTION

The present disclosure provides a compound of Formula I:

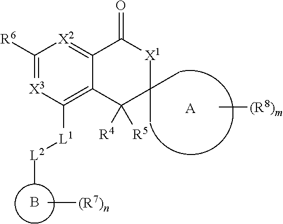

or a pharmaceutically acceptable salt thereof, wherein:
X¹ is NR¹ or O;
X² is CR² or N;
X³ is CR³ or N;
n is 0, 1, 2, 3, 4, 5, or 6;
m is 0, 1, 2, 3, 4, 5, or 6;
Ring A is C₃₋₁₄ cycloalkyl or 4-14 membered heterocycloalkyl;
Ring B is C₃₋₁₀ cycloalkyl, C₆₋₁₀ aryl, 4-10 membered heterocycloalkyl, or 5-10 membered heteroaryl;
L¹ is selected from bond, C₁₋₆ alkylene, C₁₋₆ haloalkylene, C₃₋₇ cycloalkylene, 4-7 membered heterocycloalkylene, 5-6 membered heteroarylene, —C₃₋₇ cycloalkylene-C₁₋₄ alkyl-, -(4-7 membered heterocycloalkylene)—C₁₋₄ alkyl-, -(5-6 membered heteroarylene)—C₁₋₄ alkyl-, —O—, —N(R^{L})—, —C(O)—, —C(O)N(R^{L})—, —N(R^{L})C(O)N(R^{L})—, —N(R^{L})C(O)O—, —S(O)₂—, —N(R^{L})S(O)₂—, and —N(R^{L})S(O)₂N(R^{L})—, wherein the C₁₋₆ alkylene, C₁₋₆ haloalkylene, C₃₋₇ cycloalkylene, 4-7 membered heterocycloalkylene, 5-6 membered heteroarylene, C₃₋₇ cycloalkylene-C₁₋₄ alkyl, (4-7 membered heterocycloalkylene)—C₁₋₄ alkyl, and (5-6 membered heteroarylene)—C₁₋₄ alkyl of L¹ are each optionally substituted with 1, 2, 3, or 4 independently selected R^{G} substituents;
L² is selected from bond, C₁₋₆ alkylene, C₁₋₆ haloalkylene, C₃₋₇ cycloalkylene, 4-7 membered heterocycloalkylene, 5-6 membered heteroarylene, —C₃₋₇ cycloalkylene-C₁₋₄ alkyl-, -(4-7 membered heterocycloalkylene)—C₁₋₄ alkyl-, -(5-6 membered heteroarylene)—C₁₋₄ alkyl-, —O—, —N(R^{L})—, —C(O)—, —C(O)N(R^{L})—, —N(R^{L})C(O)N(R^{L})—, —N(R^{L})C(O)O—, —S(O)₂—, —N(R^{L})S(O)₂—, and —N(R^{L})S(O)₂N(R^{L})—, wherein the C₁₋₆ alkylene, C₁₋₆ haloalkylene, C₃₋₇ cycloalkylene, 4-7 membered heterocycloalkylene, 5-6 membered heteroarylene, C₃₋₇ cycloalkylene-C₁₋₄ alkyl, (4-7 membered heterocycloalkylene)—C₁₋₄ alkyl, and (5-6 membered heteroarylene)—C₁₋₄ alkyl of L² are each optionally substituted with 1, 2, 3, or 4 independently selected R^{G} substituents;
each R^{L} is independently selected from H, C₁₋₆ alkyl, and C₁₋₆ haloalkyl;
R¹ is selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R¹ are each optionally substituted with 1, 2, 3, 4, 5, or 6 independently selected R^{G} substituents;
R² is selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a2}, —SR^{a2}—NR^{c2}R^{d2}, —C(O)R^{a2}, —C(O)OR^{a2}, —C(O)NR^{c2}R^{d2}, —C(O)NR^{c2}(OR^{a2}), —OC(O)NR^{c2}R^{d2}—NR^{c2}C(O)R^{a2}, —NR^{c2}C(O)OR^{a2}, —NR^{c2}C(O)NR^{c2}R^{d2}—NR²S(O)₂R^{b2}-R^{c2}S(O)₂, NR^{c2}R^{d2}—NR^{c2}OR^{a2}, —NR^{c2}S(O)R^{b2}, —NR^{c2}S(O)NR^{c2}R^{d2}, —S(O)R^{b2}, —S(O)₂R¹², —S(O)NR^{c2}R^{d2}, —S(O)₂NR^{c2}R^{d2}, —C(═NR^{c2})R^{a2}, —C(═NR^{c2})NR^{c2}R^{d2}—NR^{c2}C(═NR^{c2})R^{a2}—NR²C(═NR^{c2})NR^{c2}R^{d2}-R^{c2}S(O)(═NR^{c2})R^{b2}—NR^{c2}S(O)(═NR^{c2})NR²R^{d2}—S(O)(═NR^{c2})R^{b2}, and —S(O)(═NR^{c2})NR^{c2}R^{d2}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R² are each optionally substituted with 1, 2, 3, 4, 5, or 6 independently selected R^{G} substituents;
each R^{a2}, R^{b2}, R^{c2}, and R^{d2} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a2}, R^{b2}, R^{c2}, and R^{d2} are each optionally substituted with 1, 2, 3, 4, 5, or 6 independently selected R^{G} substituents; or, any R^{c2} and R^{d2} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents; and each R^{c2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl; R³ is selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a3}, —SR^{a3}—NR^{c3}R^{d3}—C(O)R³, —C(O)OR^{a3}, —C(O)NR^{c3}R^{d3}, —C(O)NR^{c3}(OR^{a3}), —OC(O)NR^{c3}R^{d3}—NR^{c3}C(O)R^{a3}, —NR^{c3}C(O)OR^{a3}, —NR^{c3}C(O)NR^{c3}R^{d3}—NR^{c3}S(O)₂R¹³, —NR^{c3}S(O)₂NR^{c3}R^{d3}—NR^{o}30R^{a3}, —NR^{c3}S(O)R^{b3}, —NR^{c3}S(O)NR^{c3}R^{d3}, —S(O)R^{b3}, —S(O)₂R¹³, —S(O)NR^{c3}R^{d3}, —S(O)₂NR^{c3}R^{d3}, —C(═NR^{c3})R^{a3}, —C(═NR^{c3})NR^{c3}R^{d3}—NR^{c3}C(═NR^{c3})R^{a3}, —NR^{c3}C(═NR^{c3})NR^{c3}R^{d3}, —NR^{c3}S(O)(═NR^{c3})R^{b3}, —NR^{c3}S(O)(═NR^{c3})NR³R^{d3}—S(O)(═NR^{c3})R^{b3}, and —S(O)(═NR^{c3})NR^{c3}R^{d3}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R³ are each optionally substituted with 1, 2, 3, 4, 5, or 6 independently selected R^{G} substituents;
each R^{a3}, R^{b3}, R^{c3}, and R^{d3} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a3}, R^{b3}, R^{c3}, and R^{d3} are each optionally substituted with 1, 2, 3, 4, 5, or 6 independently selected R^{G} substituents;
or, any R^{c3} and R^{d3} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents; and
each R^{c3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl; R⁴ is selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a4}, —SR^{a4}—NR^{c4}R^{d4}—C(O)R^{a4}, —C(O)OR^{a4}, —C(O)NR^{c4}R^{d4}, —C(O)NR^{c4}(OR^{a4}), —OC(O)NR^{c4}R^{d4}—NR^{c4}C(O)R^{a4}, —NR^{c4}C(O)OR^{a4}, —NR^{c4}C(O)NR^{c4}R^{d4}, —NR^{c4}S(O)₂R^{b4}, —NR^{c4}S(O)₂NR^{c4}R^{d4}—NR^{c4}R^{a4}, —NR^{c4}S(O)R^{b4}, —NR⁴S(O)NR^{c4}R^{d4}, —S(O)R^{b4}, —S(O)₂R^{b4}, —S(O)NR^{c4}R^{d4}, —S(O)₂NR^{c4}R^{d4}, —C(═NR^{c4})R^{a4}, —C(═NR^{c4})NR^{c4}R^{d4}—NR^{c4}C(═NR^{c4})R^{a4}, —NR^{c4}C(═NR^{c4})NR^{c4}R^{d4}, —NR^{c4}S(O)(═NR^{c4})R^{b4}, —NR^{c4}S(O)(═NR^{c4})NR^{c4}R^{d4}, —S(O)(═NR^{c4})R^{b4}, and —S(O)(═NR^{c4})NR^{c4}R^{d4}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁴ are each optionally substituted with 1, 2, 3, 4, 5, or 6 independently selected R^{G} substituents;
each R^{a4}, R^{b4}, R^{c4}, and R^{d4} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a4}, R^{b4}, R^{c4}, and R^{d4} are each optionally substituted with 1, 2, 3, 4, 5, or 6 independently selected R^{G} substituents;
or, any R^{c4} and R^{d4} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents; and
each R^{c4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
R⁵ is selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁵ are each optionally substituted with 1, 2, 3, 4, 5, or 6 independently selected R^{G} substituents;
or, R⁴ and R⁵ taken together with the carbon atom to which they are attached form a C₃₋₇ cycloalkyl, or 4-7 membered heterocycloalkyl group, wherein the C₃₋₇ cycloalkyl, and 4-7 membered heterocycloalkyl are each optionally substituted with 1, 2, 3, or 4 independently selected R^{G} substituents;
R⁶ is selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a6}, —SR^{a6}—NR⁶R^{d6}, —C(O)R^{a6}, —C(O)OR^{a6}, —C(O)NR^{c6}R^{d6}, —C(O)NR^{c6}(OR^{a6}), —OC(O)NR^{c6}R^{d6}—NR^{c6}C(O)R^{a6}, —NR⁶C(O)OR^{a6}, —NR⁶C(O)NR⁶R^{d6}—NR⁶S(O)₂R^{b6}, —NR^{c6}S(O)₂NR^{c6}R^{d6}—NR^{o}60R^{a6}, —NR^{c6}S(O)R^{b6}, —NR^{c6}S(O)NR^{c6}R^{d6}, —S(O)R^{b6}, —S(O)₂R⁶, —S(O)NR^{c6}R^{d6}, —S(O)₂NR^{c6}R^{d6}, —C(═NR^{c6})R^{a6}, —C(═NR^{c6})NR^{c6}R^{d6}—NR^{c6}C(═NR^{c6})R^{a6}—NR⁶C(═NR^{c6})NR^{c6}R^{d6}-R^{c6}S(O)(═NR^{c6})R^{b6}, —NR^{c6}S(O)(═NR^{c6})NR⁶R^{d6}—S(O)(═NR^{c6})R^{b6}, and —S(O)(═NR^{c6})NR^{c6}R^{d6}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁶ are each optionally substituted with 1, 2, 3, 4, 5, or 6 independently selected R^{G} substituents;
each R^{a6}, R^{b6}, R^{c6}, and R^{d6} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of Rae, R^{b6}, R^{c6}, and R^{d6} are each optionally substituted with 1, 2, 3, 4, 5, or 6 independently selected R^{G} substituents;
or, any R^{c6} and R^{d6} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents; and
each R^{c6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R⁷ is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a7}, —SR^{a7}, —NR^{c7}R^{d7}, —NO₂, —C(O)R^{a7}, —C(O)OR^{a7}, —C(O)NR^{c7}R^{d7}, -C(O)NR^{c7}(OR^{a7}), —OC(O)R^{a7}, —OC(O)NR^{c7}R^{d7}, —OC(O)OR^{a7}, —OS(O)₂R^{b7}, -OS(O)₂NR^{c7}R^{d7}—NR^{c7}C(O)R^{a7}, —NR^{c7}C(O)OR^{a7}, —NR⁷C(O)NR^{c7}R^{d7}—NR^{c7}S(O)₂R^{b7}—NR^{c7}S(O)₂NR⁷R^{d7}—NR^{c7}OR^{a7}, —NR^{c7}S(O)R^{b7}—NR^{c7}S(O)NR^{c7}R^{d7}—S(O)R^{b7}, —S(O)₂R^{b7}, —S(O)NR^{c7}R^{d7}, —S(O)₂NR^{c7}R^{d7}, —C(═NR^{c7})R^{a7}, —C(═NR^{c7})NR^{c7}R^{d7}, —NR^{c7}C(═NR^{c7})R^{a7}, —NR⁷C(═NR^{c7})NR^{c7}R^{d7}-R^{c7}S(O)(═NR^{c7})R^{b7}, —NR^{c7}S(O)(═NR^{c7})NR^{c7}R^{d7}—OS(O)(═NR^{c7})R^{b7}, —S(O)(═NR^{c7})R^{b7}, —S(O)(═NR^{c7})NR^{c7}R^{d7}, —C(O)NR^{c7}S(O)₂R^{b7}, —C(O)NR^{c7}S(O)₂NR^{c7}R^{d7}—S(O)₂NR^{c7}C(O)R^{b7}, —NR^{c7}S(O)NR^{c7}C(O)R^{b7}, and —P(O)R⁷R^{g7}, wherein the C₁₋₆ alkyl, C₂₋₆ alkenyl, C₂₋₆ alkynyl, C₁₋₆ haloalkyl, C₃₋₁₀ cycloalkyl, C₆0.1o aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁷ are each optionally substituted with 1, 2, 3, 4, 5, or 6 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a7}, R^{c7}, and R^{d7} are each optionally substituted with 1, 2, 3, 4, 5, or 6 independently selected R^{7A} substituents;
or, any R^{c7} and R^{d7} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b7} are each optionally substituted with 1, 2, 3, 4, 5, or 6 independently selected R^{7A} substituents;
each R^{c7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f7} and R^{g7} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{7A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a7A}, —SR^{a7A}, —NR^{c7A}, R^{d7A}, —NO₂, —C(O)R^{a7A}, —C(O)OR^{a7A}, —C(O)NR^{c7A}, R^{d7A}, —C(O)NR^{c7A}(OR^{a7A}), —OC(O)R^{a7A}, —OC(O)NR^{7A}, R^{d7A}, —OC(O)OR^{a7A}, —OS(O)₂R^{b7A}, —OS(O)₂NR^{7A}, R^{d7A}, —NR^{c7a}C(O)R^{a7A}, —NR^{c7a}C(O)OR^{a7A}, —N^{c7a}C(O)NR^{c7A}, R^{d7A}, —NR^{c7}AS(O)₂R^{b7A}, —NR^{c7}AS(O)₂NR^{c7A}, R^{d7A}, —NR^{c7A}OR^{a7A}, —NR^{c7}AS(O)R^{b7A}, —NR^{c7}AS(O)NR^{c7A}, R^{d7A}, —S(O)R^{b7A}, —S(O)₂R^{b7A}, —S(O)NR^{7A}, R^{d7A}, —S(O)₂NR^{c7A}, R^{d7A}, —C(═NR^{e7A})R^{a7A}, —C(═NR^{c7A})NR^{c7A}, R^{d7A}, —NR^{c7a}C(═NR^{c7A})R^{a7A}, —NR^{c7a}C(═NR^{c7A})NR^{7A}, R^{d7A} N^{c7}AS(O)(═NR^{c7A})R^{b7A}, —NR^{c7}AS(O)(═NR^{c7A})NR^{7A}, R^{d7A}, —OS(O)(═NR^{c7A})R^{b7A}, —S(O)(═NR^{c7A})R^{b7A}, —S(O)(═NR^{c7A})R^{c7A}, R^{d7A}, —C(O)NR^{c7}AS(O)₂R^{b7A}, —C(O)NR^{c7}AS(O)₂NR^{c7A}, R^{d7A}, —S(O)₂NR^{c7a}C(O)R^{b7A}—NR^{c7}AS(O)NR^{c7a}C(O)R^{b7A}, and —P(O)R^{f7A}R^{g7A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{7A} are each optionally substituted with 1, 2, 3, 4, 5, or 6 independently selected R^{G} substituents;
each R^{a7A}, R^{c7A}, and R^{d7A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a7A}, R^{c7A} and R^{d7A} are each optionally substituted with 1, 2, 3, 4, 5, or 6 independently selected R^{G} substituents;
or, any R^{c7A} and R^{d7A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{b7A} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b7A} are each optionally substituted with 1, 2, 3, 4, 5, or 6 independently selected R^{G} substituents;
each R^{e7A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f7A} and R^{g7A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R⁸ is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a8}, —SR^{a8}, —NR^{c8}R^{d8}, —NO₂, —C(O)R^{a8}, —C(O)OR^{a8}, —C(O)NR^{c8}R^{d8}—C(O)NR^{c8}(OR^{a8}), —OC(O)R^{a8}, —OC(O)NR^{c8}R^{d8}, —OC(O)OR^{a8}, —OS(O)₂R^{b8}, —OS(O)₂NR^{c8}R^{d8}—NR^{c8}C(O)R^{a8}, —NR^{c8}C(O)OR^{a8}, —NR^{c8}C(O)NR^{c8}R^{d8}—NR^{c8}S(O)₂R^{b8}, —NR^{c8}S(O)₂NR^{c8}R^{d8}—NR^{c8}OR^{a8}, —NRS(O)RV, —NR^{c8}S(O)NR⁸R^{d8}, —S(O)R^{b8}, —S(O)₂R^{b8}, —S(O)NR^{c8}R^{d8}, —S(O)₂NR^{c8}R^{d8}, —C(═NR^{c8})R^{a8}, —C(═NR^{c8})NR^{c8}R^{d8}—NR^{c8}C(═NR^{c8})R^{a8}, —NR⁸C(═NR^{c8})NR⁸R^{d8}R^{c8}S(O)(═NR^{c8})RH, —NR^{c8}S(O)(═NR^{c8})NR^{c8}R^{d8}—OS(O)(═NR^{c8})R^{b8}, —S(O)(═NR^{c8})R^{b8}, —S(O)(═NR^{c8})NR⁸R^{d8}, —C(O)NR^{c8}S(O)₂R″, —C(O)NR^{c8}S(O)₂NR^{c8}R^{d8}—S(O)₂NR^{c8}C(O)R^{b8}, —NR^{c8}S(O)NR^{c8}C(O)RV, and —P(O)R^{b8}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{g} are each optionally substituted with 1, 2, 3, 4, 5, or 6 independently selected R^{8A} substituents;
each R^{a8}, R^{c8}, and R^{d8} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a8}, R^{c8}, and R^{d8} are each optionally substituted with 1, 2, 3, 4, 5, or 6 independently selected R^{8A} substituents;
or, any R^{c8} and R^{d8} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{8A} substituents;
each R^{b8} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆0.1o aryl-C₁₋₄ alkyl, (4-10 membered heterocycloalkyl)-C₁₋₄ alkyl, and (5-10 membered heteroaryl)-C₁₋₄ alkyl of R^{b8} are each optionally substituted with 1, 2, 3, 4, 5, or 6 independently selected R^{8A} substituents;
each R⁸ is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R and R⁹⁸ are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{8A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a8A}, —SR^{a8A}, —NR^{e8A}, —NO₂, —C(O)R^{a8A}, —C(O)OR^{a8A}, —C(O)NR^{c8A}, R^{d8A}, —C(O)NR^{c8A}(OR^{a8A}), —OC(O)R^{a8A}, —OC(O)NR^{8A}, R^{d8A}, —OC(O)OR^{a8A}, —OS(O)₂R^{b8A}, —OS(O)₂NR^{8A}, R^{d8A}, —NR^{c8a}C(O)R^{a8A}, —NR^{c8a}C(O)OR^{a8A}, —NR^{8a}C(O)NR^{c8A}, R^{d8A}, —NR⁸AS(O)₂R^{b8A}, —R^{c8}AS(O)₂NR^{c8A}, R^{d8A}, —NR^{c8A}OR^{a8A}, —NR^{c8}AS(O)R^{b8A}, —NR⁸AS(O)NR^{c8A}, R^{d8A}, —S(O)R^{b8A}, —S(O)₂R^{b8A}, —S(O)NR^{c8A}, R^{d8A}, —S(O)₂NR^{c8A}, R^{d8A}, —C(═NR^{c8A})R^{a8A}, —C(═NR^{c8A})R^{8A}, R^{d8A}, —NR^{c8a}C(═NR^{c8A})R^{a8A}, —NR^{c8a}C(═NR^{e8A})NR^{c8A}, R^{d8A}, —R⁸AS(O)(═NR^{c8A})R^{b8A}, —NR^{c8}AS(O)(═NR^{c8A})NR^{c8A}, R^{d8A}, —OS(O)(═NR^{c8A})R^{b8A}, —S(O)(═NR^{c8A})R^{b8A}, —S(O)(═NR^{c8A})R^{8A}, R^{d8A}, —C(O)NR^{c8}AS(O)₂R^{b8A}, —C(O)NR⁸AS(O)₂NR^{c8A}, R^{d8A}, —S(O)₂NR^{c8a}C(O)R^{b8A}, —NR^{c8}AS(O)NR^{c8a}C(O)R^{b8A}, and —P(O)R^{f}AR^{g8A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{8A} are each optionally substituted with 1, 2, 3, 4, 5, or 6 independently selected R^{G} substituents;
each R^{a8A}, R^{b8A}, and R^{d8A}, is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a8A}, R^{c8}A and R^{d8A}, are each optionally substituted with 1, 2, 3, 4, 5, or 6 independently selected R^{G} substituents;
or, any R^{c8A} and R^{d8A}, attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{b8A} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b8A} are each optionally substituted with 1, 2, 3, 4, 5, or 6 independently selected R^{G} substituents;
each R^{c8A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f8A} and R^{g8A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{G} is independently selected from OH, CN, halo, oxo, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, C₁₋₄ alkoxy-C₁₋₄ alkyl, C₃₋₇ cycloalkyl, 4-7 membered heterocycloalkyl, C₁₋₄ alkoxy, C₁₋₄ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, aminocarbonyloxy, C₁₋₃ alkylaminocarbonyloxy, di(C₁₋₃ alkyl)aminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, X¹ is NR¹.

In some embodiments, R¹ is selected from H, C₁₋₆ alkyl, C₂₋₆ alkenyl, C₂₋₆ alkynyl, and C₁₋₆ haloalkyl.

In some embodiments, R¹ is selected from H, C₁₋₆ alkyl, and C₁₋₆ haloalkyl.

In some embodiments, R¹ is selected from C₁₋₆ alkyl and C₁₋₆ haloalkyl.

In some embodiments, R¹ is C₁₋₆ alkyl.

In some embodiments, R¹ is C₁₋₃ alkyl.

In some embodiments, R¹ is methyl.

In some embodiments, X¹ is NCH₃.

In some embodiments, X¹ is O.

In some embodiments, X² is CR².

In some embodiments, R² is selected from H, C₁₋₆ alkyl, C₂₋₆ alkenyl, C₂₋₆ alkynyl, and C₁₋₆ haloalkyl.

In some embodiments, R² is selected from H or C₁₋₆ alkyl.

In some embodiments, R² is H.

In some embodiments, X² is CH.

In some embodiments, X² is N.

In some embodiments, X³ is CR³.

In some embodiments, R³ is selected from H, C₁₋₆ alkyl, C₂₋₆ alkenyl, C₂₋₆ alkynyl, and C₁₋₆ haloalkyl.

In some embodiments, R³ is selected from H and C₁₋₆ alkyl.

In some embodiments, R³ is H.

In some embodiments, X³ is CH.

In some embodiments, X³ is N.

In some embodiments, R⁴ is selected from H, halo, C₁₋₆ alkyl, C₂₋₆ alkenyl, C₂₋₆ alkynyl, and C₁₋₆ haloalkyl.

In some embodiments, R⁴ is selected from H and C₁₋₆ alkyl.

In some embodiments, R⁴ is H.

In some embodiments, R⁵ is selected from H, halo, C₁₋₆ alkyl, C₂₋₆ alkenyl, C₂₋₆ alkynyl, and C₁₋₆ haloalkyl.

In some embodiments, R⁵ is selected from H and C₁₋₆ alkyl.

In some embodiments, R⁵ is H.

In some embodiments, R⁴ and R⁵ are each independently H or C₁₋₆ alkyl.

In some embodiments, R⁴ and R⁵ are each H.

In some embodiments, R², R³, R⁴, and R⁵ are each H.

In some embodiments, R², R⁴, and R⁵ are each H.

In some embodiments, R³, R⁴, and R⁵ are each H.

In some embodiments, R⁶ is selected from H, halo, C₁₋₆ alkyl, C₂₋₆ alkenyl, C₂₋₆ alkynyl, and C₁₋₆ haloalkyl.

In some embodiments, R⁶ is selected from H, C₁₋₆ alkyl, and C₁₋₆ haloalkyl.

In some embodiments, R⁶ is selected from H and C₁₋₆ alkyl.

In some embodiments, R⁶ is selected from H and C₁₋₃ alkyl.

In some embodiments, R⁶ is C₁₋₆ alkyl.

In some embodiments, R⁶ is C₁₋₃ alkyl.

In some embodiments, R⁶ is methyl.

In some embodiments, Ring A is C₃₋₁₄ cycloalkyl.

In some embodiments, Ring A is C₇₋₁₄ cycloalkyl.

In some embodiments, Ring A is C₇₋₁₂ cycloalkyl.

In some embodiments, Ring A is a bicyclic C₇₋₁₄ cycloalkyl.

In some embodiments, Ring A is bicyclic C₇₋₁₂ cycloalkyl.

In some embodiments, Ring A is a C₉ cycloalkyl.

In some embodiments, Ring A is a bicyclic C₉ cycloalkyl.

In some embodiments, Ring A is dihydroindenyl.

In some embodiments, m is 0, 1, 2, 3, or 4.

In some embodiments, m is 0, 1, 2, or 3.

In some embodiments, m is 0, 1, or 2.

In some embodiments, m is 0 or 1.

In some embodiments, m is 0.

In some embodiments, Ring A is

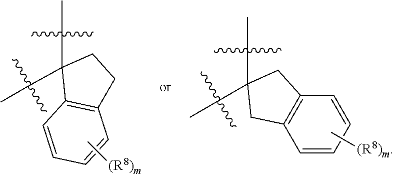

In some embodiments, Ring A is

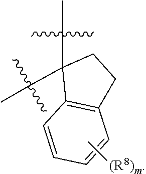

In some embodiments, Ring A is

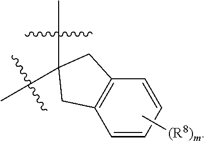

In some embodiments, Ring A is

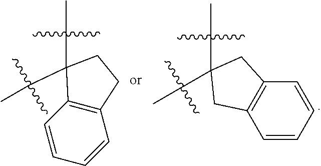 [SMILES: *C1(*)CCc2ccccc21 ; *C1(*)Cc2ccccc2C1]

In some embodiments, Ring A is

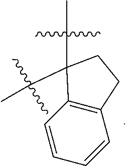 [SMILES: *C1(*)CCc2ccccc21]

In some embodiments, Ring A is

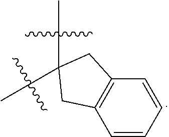 [SMILES: *C1(*)Cc2ccccc2C1]

In some embodiments, L¹ is selected from bond, C₁₋₆ alkylene, C₁₋₆ haloalkylene, C₃₋₇ cycloalkylene, 4-7 membered heterocycloalkylene, and 5-6 membered heteroarylene.

In some embodiments, L¹ is selected from bond, C₁₋₆ alkylene, and C₁₋₆ haloalkylene.

In some embodiments, L¹ is selected from C₁₋₆ alkylene and C₁₋₆ haloalkylene.

In some embodiments, L¹ is selected from C₁₋₃ alkylene and C₁₋₃ haloalkylene.

In some embodiments, L¹ is C₁₋₆ alkylene.

In some embodiments, L¹ is C₁₋₃ alkylene.

In some embodiments, L¹ is —CH(CH₃)—.

In some embodiments, L² is selected from —O—, —N(R^{L})—, —C(O)—, —C(O)N(R^{L})—, —N(R^{L})C(O)N(R^{L})—, —N(R^{L})C(O)O—, —S(O)₂—, —N(R^{L})S(O)₂—, and —N(R^{L})S(O)₂N(R^{L})—.

In some embodiments, L² is selected from —N(R^{L})—, —C(O)—, and —C(O)N(R^{L})—.

In some embodiments, L² is —N(R^{L})—.

In some embodiments, each R^{L} is independently selected from H and C₁₋₆ alkyl.

In some embodiments, each R^{L} is H.

In some embodiments, L² is —NH—.

In some embodiments, L¹-L² forms—C₁₋₆ alkylene-N(R^{L})—.

In some embodiments, L¹-L² forms —CH(CH₃)—N(R^{L})—.

In some embodiments, L¹-L² forms —CH(CH₃)—NH—.

In some embodiments, Ring B is C₃₋₇ cycloalkyl, phenyl, 4-7 membered heterocycloalkyl, or 5-6 membered heteroaryl.

In some embodiments, Ring B is C₆₋₁₀ aryl or 5-10 membered heteroaryl.

In some embodiments, Ring B is C₆₋₁₀ aryl.

In some embodiments, Ring B is 5-10 membered heteroaryl.

In some embodiments, Ring B is 5-6 membered heteroaryl.

In some embodiments, Ring B is phenyl or 5-6 membered heteroaryl.

In some embodiments, Ring B is phenyl or pyridinyl.

In some embodiments, Ring B is phenyl.

In some embodiments, Ring B is pyridinyl.

In some embodiments, n is 0, 1, 2, 3, or 4.

In some embodiments, n is 0, 1, 2, or 3.

In some embodiments, n is 1, 2, or 3.

In some embodiments, n is 1 or 2.

In some embodiments, n is 1.

In some embodiments, n is 2.

In some embodiments, each R⁷ is independently selected from halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, CN, —OR^{a7}, —NR^{c7}R^{d7}, —C(O)R^{a7}, —C(O)OR^{a7}, and —C(O)NR^{c7}R^{d7}, wherein the C₁₋₆ alkyl, C₂₋₆ alkenyl, C₂₋₆ alkynyl, C₃₋₁₀ cycloalkyl, C₆₋₁₀ aryl, 4-10 membered heterocycloalkyl, and 5-10 membered heteroaryl are each optionally substituted with 1, 2, 3, or 4 independently selected R^{7A} substituents.

In some embodiments, each R^{a7}, R^{b7}, R^{c7}, and R^{d7} is independently selected from H, C₁₋₆ alkyl and C₁₋₆ haloalkyl.

In some embodiments, each R^{a7}, R^{c7}, and R^{d7} is independently selected from H, C₁₋₆ alkyl and C₁₋₆ haloalkyl.

In some embodiments, each R⁷ is independently selected from halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, CN, —OR^{a7}, —NR^{c7}R^{d7}, —C(O)R^{a7}, —C(O)OR^{a7}, and —C(O)NR^{c7}R^{d7}, wherein the C₁₋₆ alkyl, C₂₋₆ alkenyl, C₂₋₆ alkynyl, C₃₋₁₀ cycloalkyl, C₆₋₁₀ aryl, 4-10 membered heterocycloalkyl, and 5-10 membered heteroaryl are each optionally substituted with 1, 2, 3, or 4 independently selected R^{7A} substituents; and each R^{a7}, R^{c7}, and R^{d7} is independently selected from H, C₁₋₆ alkyl and C₁₋₆ haloalkyl.

In some embodiments, each R⁷ is independently selected from halo, C₁₋₆ alkyl, C₁₋₆ haloalkyl, C₃₋₁₀ cycloalkyl, C₆₋₁₀ aryl, 4-10 membered heterocycloalkyl, 5-10 membered heteroaryl, —C(O)R^{a7}, —C(O)OR^{a7}, and —C(O)NR^{c7}R^{d7}, wherein the C₁₋₆ alkyl, C₁₋₆ haloalkyl, C₃₋₁₀ cycloalkyl, C₆₋₁₀ aryl, 4-10 membered heterocycloalkyl, and 5-10 membered heteroaryl are each optionally substituted with 1, 2, 3, or 4 independently selected R^{7A} substituents.

In some embodiments, each R⁷ is independently selected from halo, C₁₋₆ alkyl, C₁₋₆ haloalkyl, C₃₋₁₀ cycloalkyl, C₆₋₁₀ aryl, 4-10 membered heterocycloalkyl, 5-10 membered heteroaryl, —C(O)R^{a7}, —C(O)OR^{a7}, and —C(O)NR^{c7}R^{d7}, wherein the C₁₋₆ alkyl, C₁₋₆ haloalkyl, C₃₋₁₀ cycloalkyl, C₆₋₁₀ aryl, 4-10 membered heterocycloalkyl, and 5-10 membered heteroaryl are each optionally substituted with 1, 2, 3, or 4 independently selected R^{7A} substituents; and each R^{a7}, R^{c7}, and R^{d7} is independently selected from H, C₁₋₆ alkyl and C₁₋₆ haloalkyl.

In some embodiments, each R⁷ is independently selected from halo, C₁₋₆ alkyl, 5-6 membered heteroaryl, and —C(O)OR^{a7}, wherein the 5-6 membered heteroaryl is optionally substituted with 1, 2, 3, or 4 independently selected R^{7A} substituents.

In some embodiments, each R⁷ is independently selected from halo, C₁₋₆ alkyl, 5-6 membered heteroaryl, and —C(O)OR^{a7}, wherein the 5-6 membered heteroaryl is optionally substituted with 1, 2, 3, or 4 independently selected R^{7A} substituents; and each R^{a7} is independently selected from H and C₁₋₆ alkyl.

In some embodiments, each R⁷ is independently selected from halo, C₁₋₃ alkyl, 5-6 membered heteroaryl, and —C(O)OR^{a7}, wherein the C₁₋₃ alkyl and 5-6 membered heteroaryl are each optionally substituted with 1, 2, 3, or 4 independently selected R^{7A} substituents.

In some embodiments, each R⁷ is independently selected from halo, C₁₋₃ alkyl, 5-6 membered heteroaryl, and —C(O)OR^{a7}, wherein the C₁₋₃ alkyl and 5-6 membered heteroaryl are each optionally substituted with 1, 2, 3, or 4 independently selected R^{7A} substituents; and each R^{a7} is independently selected from H and C₁₋₆ alkyl.

In some embodiments, each R⁷ is independently selected from chloro, methyl, pyrazolyl, pyridinyl, and —C(O)OR^{a7}, wherein the methyl, pyrazolyl, and pyridinyl are each optionally substituted with 1, 2, 3, or 4 independently selected R^{7A} substituents.

In some embodiments, each R⁷ is independently selected from chloro, methyl, pyrazolyl, pyridinyl, and —C(O)OR^{a7}, wherein the methyl, pyrazolyl, and pyridinyl are each optionally substituted with 1, 2, 3, or 4 independently selected R^{7A} substituents; and each R^{a7} is independently selected from H and C₁₋₆ alkyl.

In some embodiments, each R^{7A} is independently selected from oxo, halo, C₁₋₆ alkyl, C₁₋₆ haloalkyl, C₂₋₆ alkenyl, and C₂₋₆ alkynyl.

In some embodiments, each R^{7A} is independently selected from oxo, halo, C₁₋₆ alkyl, and C₁₋₆ haloalkyl.

In some embodiments, each R^{7A} is independently selected from halo and C₁₋₆ alkyl.

In some embodiments, each R^{7A} is independently selected from halo and C₁₋₃ alkyl.

In some embodiments, each R^{7A} is independently selected from fluoro and methyl.

In some embodiments, each R⁷ is independently selected from halo, C₁₋₆ alkyl, C₁₋₆ haloalkyl, C₃₋₁₀ cycloalkyl, C₆₋₁₀ aryl, 4-10 membered heterocycloalkyl, 5-10 membered heteroaryl, —C(O)R^{a7}, —C(O)OR^{a7}, and —C(O)NR^{c7}R^{d7}, wherein the C₁₋₆ alkyl, C₁₋₆ haloalkyl, C₃₋₁₀ cycloalkyl, C₆₋₁₀ aryl, 4-10 membered heterocycloalkyl, and 5-10 membered heteroaryl are each optionally substituted with 1, 2, 3, or 4 independently selected R^{7A} substituents;
each R^{a7}, R^{c7}, and R^{d7} is independently selected from H, C₁₋₆ alkyl and C₁₋₆ haloalkyl; and
each R^{7A} is independently selected from oxo, halo, C₁₋₆ alkyl, C₁₋₆ haloalkyl, C₂₋₆ alkenyl, and C₂₋₆ alkynyl.

In some embodiments, each R⁷ is independently selected from halo, C₁₋₆ alkyl, 5-6 membered heteroaryl, and —C(O)OR^{a7}, wherein the 5-6 membered heteroaryl is optionally substituted with 1, 2, 3, or 4 independently selected R^{7A} substituents;
each R^{a7} is independently selected from H and C₁₋₆ alkyl; and
each R^{7A} is independently selected from oxo, halo, C₁₋₆ alkyl, C₁₋₆ haloalkyl, C₂₋₆ alkenyl, and C₂₋₆ alkynyl.

In some embodiments, each R⁷ is independently selected from halo, C₁₋₃ alkyl, 5-6 membered heteroaryl, and —C(O)OR^{a7}, wherein the C₁₋₃ alkyl and 5-6 membered heteroaryl are each optionally substituted with 1, 2, 3, or 4 independently selected R^{7A} substituents;
each R^{a7} is independently selected from H and C₁₋₆ alkyl; and
each R^{7A} is independently selected from halo and C₁₋₆ alkyl.

In some embodiments, each R⁷ is independently selected from chloro, methyl, pyrazolyl, pyridinyl, and —C(O)OR^{a7}, wherein the methyl, pyrazolyl, and pyridinyl are each optionally substituted with 1, 2, 3, or 4 independently selected R^{7A} substituents; each R^{a7} is independently selected from H and C₁₋₆ alkyl; and each R^{7A} is independently selected from halo and C₁₋₆ alkyl.

In some embodiments, each R⁷ is independently selected from chloro, methyl, COOH, fluoropyridinyl, and methylpyrazolyl.

In some embodiments:
X¹ is NR¹ or O;
X² is CR² or N;
X³ is CR³ or N;
n is 0, 1, 2, 3, or 4;
m is 0, 1, 2, 3, or 4;
Ring A is C₃₋₁₄ cycloalkyl or 4-14 membered heterocycloalkyl;
Ring B is C₃₋₁₀ cycloalkyl, C₆₋₁₀ aryl, 4-10 membered heterocycloalkyl, or 5-10 membered heteroaryl;
L¹ is selected from bond, C₁₋₆ alkylene, and C₁₋₆ haloalkylene;
L² is selected from —N(R^{L})—, —C(O)—, and —C(O)N(R^{L})—;
each R^{L} is independently selected from H, C₁₋₆ alkyl, and C₁₋₆ haloalkyl;
R¹ is selected from H, C₁₋₆ alkyl, C₂₋₆ alkenyl, C₂₋₆ alkynyl, and C₁₋₆ haloalkyl;
R² is selected from H, C₁₋₆ alkyl, C₂₋₆ alkenyl, C₂₋₆ alkynyl, and C₁₋₆ haloalkyl;
R³ is selected from H, C₁₋₆ alkyl, C₂₋₆ alkenyl, C₂₋₆ alkynyl, and C₁₋₆ haloalkyl;
R⁴ is selected from H, halo, C₁₋₆ alkyl, C₂₋₆ alkenyl, C₂₋₆ alkynyl, and C₁₋₆ haloalkyl;
R⁵ is selected from H, halo, C₁₋₆ alkyl, C₂₋₆ alkenyl, C₂₋₆ alkynyl, and C₁₋₆ haloalkyl;
R⁶ is selected from H, halo, C₁₋₆ alkyl, C₂₋₆ alkenyl, C₂₋₆ alkynyl, and C₁₋₆ haloalkyl;
each R⁷ is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a7}, —SR^{a7}, —NR^{c7}R^{d7}, —NO₂, —C(O)R^{a7}, —C(O)OR^{a7}, —C(O)NR^{c7}R^{d7}, —C(O)NR^{c7}(OR^{a7}), —OC(O)R^{a7}, —OC(O)NR^{c7}R^{d7}, —OC(O)OR^{a7}, —OS(O)₂R^{b7}, —OS(O)₂NR^{c7}R^{d7}—NR^{c7}C(O)R^{a7}—NR^{c7}C(O)OR^{a7}, —NR⁷C(O)NR^{c7}R^{d7}—NR⁷S(O)₂R^{b7}—NR⁷S(O)₂NR^{c7}R^{d7}—NR^{o}70R^{a7}, —NR^{c7}S(O)R^{b7}, —NR^{c7}S(O)NR^{c7}R^{d7}, —S(O)R^{b7}, —S(O)₂R^{b7}, —S(O)NR^{c7}R^{d7}, and —S(O)₂NR⁷R^{d7}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁷ are each optionally substituted with 1, 2, 3, 4, 5, or 6 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
or, any R^{c7} and R^{d7} attached to the same N atom, together with the N atom to which they are attached, form a 4-10 membered heterocycloalkyl group;
each R^{b7} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{7A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a7A}, —SR^{a7A}, —N^{c7A}, R^{d7A}, —NO₂, —C(O)R^{a7A}, —C(O)OR^{a7A}, —C(O)NR^{c7A}, R^{d7A}, —C(O)NR^{c7A}(OR^{a7A}), —OC(O)R^{a7A}, —OC(O)NR^{c7A}, R^{d7A}, —OC(O)OR^{a7A}, —OS(O)₂R^{b7A}, —OS(O)₂NR^{7A}, R^{d7A}, —NR^{c7a}C(O)R^{a7A}, —NR^{c7a}C(O)OR^{a7A}, —NR^{c7a}C(O)NR^{c7A}, R^{d7A}, —NR^{c7}AS(O)₂R^{b7A}, —NR^{c7}AS(O)₂NR^{c7A}, R^{d7A}, —NR^{c7A}OR^{a7A}, —NR^{c7}AS(O)R^{b7A}, —NR^{c7}AS(O)NR^{c7A}, R^{d7A}, —S(O)R^{b7A}, and —S(O)₂R^{b7}A;
each R^{a7A}, R^{c7A}, and R^{d7A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
or, any R^{c7A} and R^{d7A} attached to the same N atom, together with the N atom to which they are attached, form a 4-10 membered heterocycloalkyl group;
each R^{b7A} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl; and
each R^{f7A} is independently selected from selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, and —CN.

In some embodiments:
X¹ is NR¹ or O;
X² is CR² or N;
X³ is CR³ or N;
n is 0, 1, 2, 3, or 4;
m is 0, 1, 2, 3, or 4;
Ring A is C₃₋₁₄ cycloalkyl or 4-14 membered heterocycloalkyl;
Ring B is C₃₋₁₀ cycloalkyl, C₆₋₁₀ aryl, 4-10 membered heterocycloalkyl, or 5-10 membered heteroaryl;
L¹ is selected from bond, C₁₋₆ alkylene, and C₁₋₆ haloalkylene;
L² is selected from —N(R^{L})—, —C(O)—, and —C(O)N(R^{L})—;
each R^{L} is independently selected from H, C₁₋₆ alkyl, and C₁₋₆ haloalkyl;
R¹ is selected from H, C₁₋₆ alkyl, C₂₋₆ alkenyl, C₂₋₆ alkynyl, and C₁₋₆ haloalkyl;
R² is selected from H, C₁₋₆ alkyl, C₂₋₆ alkenyl, C₂₋₆ alkynyl, and C₁₋₆ haloalkyl;
R³ is selected from H, C₁₋₆ alkyl, C₂₋₆ alkenyl, C₂₋₆ alkynyl, and C₁₋₆ haloalkyl;
R⁴ is selected from H, halo, C₁₋₆ alkyl, C₂₋₆ alkenyl, C₂₋₆ alkynyl, and C₁₋₆ haloalkyl;
R⁵ is selected from H, halo, C₁₋₆ alkyl, C₂₋₆ alkenyl, C₂₋₆ alkynyl, and C₁₋₆ haloalkyl;
R⁶ is selected from H, halo, C₁₋₆ alkyl, C₂₋₆ alkenyl, C₂₋₆ alkynyl, and C₁₋₆ haloalkyl;
each R⁷ is independently selected from halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, CN, —OR^{a7}, —NR^{c7}R^{d7}, —C(O)R^{a7}, —C(O)OR^{a7}, and -C(O)NR^{c7}R^{d7}, wherein the C₁₋₆ alkyl, C₂₋₆ alkenyl, C₂₋₆ alkynyl, C₃₋₁₀ cycloalkyl, C₆₋₁₀ aryl, 4-10 membered heterocycloalkyl, and 5-10 membered heteroaryl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{7A} is independently selected from oxo, halo, C₁₋₆ alkyl, C₁₋₆ haloalkyl, C₂₋₆ alkenyl, and C₂₋₆ alkynyl; and
each R^{f7A} is independently selected from selected from oxo, halo, C₁₋₆ alkyl, C₂₋₆ alkenyl, C₂₋₆ alkynyl, and C₁₋₆ haloalkyl.

In some embodiments, the compound of Formula I is a compound of Formula II:

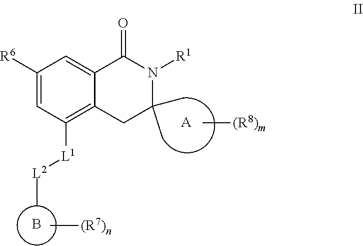

or a pharmaceutically acceptable salt thereof.

In some embodiments, the compound of Formula I is a compound of Formula III:

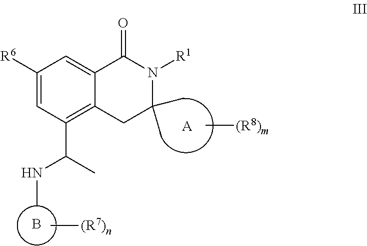

or a pharmaceutically acceptable salt thereof.

In some embodiments, the compound of Formula I is a compound of Formula

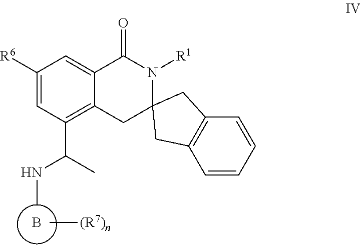

or a pharmaceutically acceptable salt thereof.

In some embodiments, the compound of Formula I is a compound of Formula

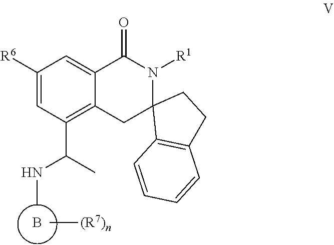

or a pharmaceutically acceptable salt thereof.

In some embodiments, the compound of Formula I is a compound of Formula VI:

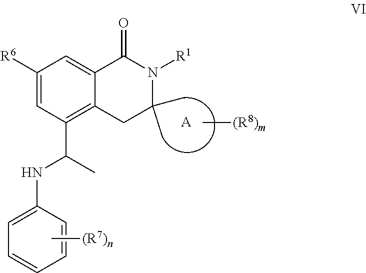

or a pharmaceutically acceptable salt thereof.

In some embodiments, the compound of Formula I is a compound of Formula VII:

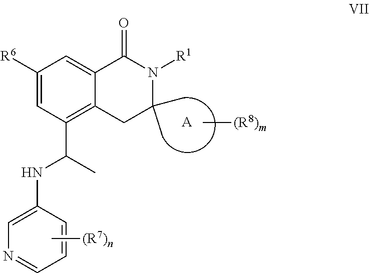

or a pharmaceutically acceptable salt thereof.

In some embodiments, the compound provided herein is selected from:
2-[1-(2,7-dimethyl-1-oxo-spiro[4H-isoquinoline-3,1′-indane]-5yl)ethylamino]benzoic acid;
3-[1-(2,7-dimethyl-1-oxo-spiro[4H-isoquinoline-3,1′-indane]-5-yl)ethylamino]-6-methyl-pyridine-2-carboxylic acid;
6-chloro-3-[1-(2,7-dimethyl-1-oxo-spiro[4H-isoquinoline-3,1′-indane]-5-yl)ethylamino]pyridine-2-carboxylic acid;
2-[1-(2,7-dimethyl-1-oxo-spiro[4H-isoquinoline-3,2′-indane]-5-yl)ethylamino]benzoic acid;
3-((1-(2′,7′-dimethyl-1′-oxo-1,1′,3,4′-tetrahydro-2′H-spiro[indene-2,3′-isoquinolin]-5′-yl)ethyl)amino)-6-methylpicolinic acid;
5-[1-[[6-chloro-2-(1-methylpyrazol-4-yl)-3-pyridyl]amino]ethyl]-2,7-dimethyl-spiro[4H-isoquinoline-3,1′-indane]-1-one; and
5-[1-[[6-chloro-2-(3-fluoro-4-pyridyl)-3-pyridyl]amino]ethyl]-2,7-dimethyl-spiro[4H-isoquinoline-3,1′-indane]-1-one;
   or a pharmaceutically acceptable salt thereof.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, divalent linking substituents are described. It is specifically intended that each divalent linking substituent include both the forward and backward forms of the linking substituent. For example, —NR(CR′R″)ₙ—includes both —NR(CR′R″)ₙ—and —(CR′R″)~NR—. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.”

Throughout the definitions, the terms “Cₙ₋ₘ” and “Cₘ₋ₙ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, from 2 to 6 carbon atoms, from 2 to 4 carbon atoms, from 2 to 3 carbon atoms, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, the term “aryl,” employed alone or in combination with other terms, refers to an aromatic hydrocarbon group, which may be monocyclic or polycyclic (e.g., having 2, 3 or 4 fused rings). The term “Cn-m aryl” refers to an aryl group having from n to m ring carbon atoms. Aryl groups include, e.g., phenyl, naphthyl, anthracenyl, phenanthrenyl, and the like. In some embodiments, aryl groups have from 5 to 10 carbon atoms. In some embodiments, the aryl group is phenyl or naphthyl. In some embodiments, the aryl is phenyl.

As used herein, “halo” refers to F, Cl, Br, or I. In some embodiments, a halo is F, Cl, or Br. In some embodiments, a halo is F or Cl. In some embodiments, a halo is F. In some embodiments, a halo is Cl.

As used herein, “Cn-m haloalkoxy” refers to a group of formula —O-haloalkyl having n to m carbon atoms. Example haloalkoxy groups include OCF₃ and OCHF₂.

In some embodiments, the haloalkoxy group is fluorinated only. In some embodiments, the alkyl group has 1 to 6, 1 to 4, or 1 to 3 carbon atoms.

As used herein, the term “Cn-m haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like.

As used herein, “cycloalkyl” refers to non-aromatic cyclic hydrocarbons including cyclized alkyl and alkenyl groups. Cycloalkyl groups can include mono- or polycyclic (e.g., having 2 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl.

As used herein, “heteroaryl” refers to a monocyclic or polycyclic (e.g., having 2 fused rings) aromatic heterocycle having at least one heteroatom ring member selected from N, O, S and B. In some embodiments, the heteroaryl ring has 1, 2, 3, or 4 heteroatom ring members independently selected from N, O, S and B. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, S, and B. In some embodiments, the heteroaryl is a 5-, 7-, 8-, 9-, or 10-membered monocyclic or bicyclic heteroaryl having 1, 2, 3, or 4 heteroatom ring members independently selected from N, O, S, and B. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-, 7-, 8-, 9-, or 10-membered monocyclic or bicyclic heteroaryl having 1, 2, 3, or 4 heteroatom ring members independently selected from N, O, and S. In some embodiments, the heteroaryl is a 5-6 membered monocyclic heteroaryl having 1 or 2 heteroatom ring members independently selected from N, O, S, and B. In some embodiments, the heteroaryl is a 5 membered monocyclic heteroaryl having 1 or 2 heteroatom ring members independently selected from N, O, S, and B. In some embodiments, the heteroaryl is a 5 membered monocyclic heteroaryl having 1 or 2 heteroatom ring members independently selected from N, O, and S. In some embodiments, the heteroaryl group contains 5 to 10, 5 to 7, 3 to 7, or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, thienyl (or thiophenyl), furyl (or furanyl), pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4-oxadiazolyl and 1,2-dihydro-1,2-azaborine, pyridinyl, pyrimidinyl, pyrazinyl, pyridazinyl, azolyl, triazolyl, thiadiazolyl, quinolinyl, isoquinolinyl, indolyl, benzothiophenyl, benzofuranyl, benzisoxazolyl, imidazo[1,2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, triazolo[4,3-a]pyridinyl, 1H-pyrrolo[3,2-b]pyridinyl, 1H-pyrrolo[2,3-b]pyridinyl, pyrazolo[1,5-a]pyridinyl, indazolyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, S, and B, and wherein the ring-forming carbon atoms and heteroatoms of a heterocycloalkyl group can be optionally substituted by one or more oxo or sulfido (e.g., C(0), S(0), C(S), or S(O)₂, etc.).

When a ring-forming carbon atom or heteroatom of a heterocycloalkyl group is optionally substituted by one or more oxo or sulfide, the O or S of said group is in addition to the number of ring-forming atoms specified herein (e.g., a 1-methyl-6-oxo-1,6-dihydropyridazin-3-yl is a 6-membered heterocycloalkyl group, wherein a ring-forming carbon atom is substituted with an oxo group, and wherein the 6-membered heterocycloalkyl group is further substituted with a methyl group).

Heterocycloalkyl groups include monocyclic and polycyclic (e.g., having 2 fused rings) systems. Included in heterocycloalkyl are monocyclic and polycyclic 3 to 10, 4 to 10, 5 to 10, 4 to 7, 5 to 7, or 5 to 6 membered heterocycloalkyl groups.

Heterocycloalkyl groups can also include spirocycles and bridged rings (e.g., a 5 to 10 membered bridged biheterocycloalkyl ring having one or more of the ring-forming carbon atoms replaced by a heteroatom independently selected from N, O, S, and B).

The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring.

In some embodiments, the heterocycloalkyl group contains 3 to 10 ring-forming atoms, 4 to 10 ring-forming atoms, 4 to 8 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S and B and having one or more oxidized ring members. In some embodiments, the heterocycloalkyl is a monocyclic or bicyclic 5-10 membered heterocycloalkyl having 1, 2, 3, or 4 heteroatoms independently selected from N, O, S, and B and having one or more oxidized ring members. In some embodiments, the heterocycloalkyl is a monocyclic or bicyclic 5 to 10 membered heterocycloalkyl having 1, 2, 3, or 4 heteroatoms independently selected from N, O, and S and having one or more oxidized ring members. In some embodiments, the heterocycloalkyl is a monocyclic 5 to 6 membered heterocycloalkyl having 1, 2, 3, or 4 heteroatoms independently selected from N, O, and S and having one or more oxidized ring members.

Example heterocycloalkyl groups include pyrrolidin-2-one (or 2-oxopyrrolidinyl), 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, 1,2,3,4-tetrahydroisoquinoline, tetrahydrothiopheneyl, tetrahydrothiopheneyl 1,1-dioxide, benzazapene, azabicyclo[3.1.0]hexanyl, diazabicyclo[3.1.0]hexanyl, oxobicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxobicyclo[2.2.2]octanyl, azabicyclo[2.2.2]octanyl, azaadamantanyl, diazaadamantanyl, oxo-adamantanyl, azaspiro[3.3]heptanyl, 2-azaspiro[3.3]heptanyl, diazaspiro[3.3]heptanyl, azaspiro[3.5]nonanyl, 7-azaspiro[3.5]nonanyl, oxo-azaspiro[3.3]heptanyl, azaspiro[3.4]octanyl, diazaspiro[3.4]octanyl, oxo-azaspiro[3.4]octanyl, azaspiro[2.5]octanyl, diazaspiro[2.5]octanyl, azaspiro[4.4]nonanyl, diazaspiro[4.4]nonanyl, oxo-azaspiro[4.4]nonanyl, azaspiro[4.5]decanyl, diazaspiro[4.5]decanyl, diazaspiro[4.4]nonanyl, oxo-diazaspiro[4.4]nonanyl, oxo-dihydropyridazinyl, oxo-2,6-diazaspiro[3.4]octanyl, oxohexahydropyrrolo[1,2-a]pyrazinyl, 3-oxopiperazinyl, oxo-pyrrolidinyl, oxo-pyridinyl, and the like.

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms.

As used herein “Cₒ₋ₚ aryl-Cₙ₋ₘ alkyl-” refers to a group of formula aryl-alkylene-, wherein the aryl has o to p carbon atoms and the alkylene linking group has n to m carbon atoms.

As used herein, “heteroaryl-Cₙ₋ₘ alkyl-” refers to a group of formula heteroaryl-alkylene-, wherein alkylene linking group has n to m carbon atoms.

As used herein “heterocycloalkyl-Cₙ₋ₘ alkyl-” refers to a group of formula heterocycloalkyl-alkylene-, wherein alkylene linking group has n to m carbon atoms.

As used herein, an “alkyl linking group” or “alkylene linking group” is a bivalent straight chain or branched alkyl linking group (“alkylene group”). For example, “Cₒ₋ₚ cycloalkyl-Cₙ₋ₘ alkyl-”, “C_{o-P} aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-dilyl, propan-1,2-diyl, propan-1,1-diyl and the like.

As used herein, a “haloalkyl linking group” or “haloalkylene linking group” is a bivalent straight chain or branched haloalkyl linking group (“haloalkylene group”).

Example haloalkylene groups include —CF₂—, —C₂F₄—, —CHF—, —CCl₂—, —CHCl—, —C₂Cl₄—, and the like.

As used herein, a “cycloalkyl linking group” or “cycloalkylene linking group” is a bivalent straight chain or branched cycloalkyl linking group (“cycloalkylene group”). Examples of “cycloalkyl linking groups” or “cycloalkylene groups” include cyclopropy-1,1,-diyl, cyclopropy-1,2-diyl, cyclobut-1,3,-diyl, cyclopent-1,3,-diyl, cyclopent-1,4,-diyl, cyclohex-1,2,-diyl, cyclohex-1,3,-diyl, cyclohex-1,4,-diyl, and the like.

As used herein, a “heterocycloalkyl linking group” or “heterocycloalkylene linking group” is a bivalent straight chain or branched heterocycloalkyl linking group (“heterocycloalkylene group”). Examples of “heterocycloalkyl linking groups” or “heterocycloalkylene groups” include azetidin-1,2-diyl, azetidin-1,3-diyl, pyrrolidin-1,2-diyl, pyrrolidin-1,3-diyl, pyrrolidin-2,3-diyl, piperidin-1,2-diyl, piperidin-1,3-diyl, piperidin-1,4-diyl, piperidin-2,3-diyl, piperidin-2,4-diyl, and the like.

As used herein, a “heteroaryl linking group” or “heteroarylene linking group” is a bivalent straight chain or branched heteroaryl linking group (“heteroarylene group”). Examples of “heteroaryl linking groups” or “heteroarylene groups” include pyrazol-1,3-diyl, imidazol-1,2,-diyl, pyridin-2,3-diyl, pyridin-2,4-diyl, pyridin-3,4-diyl, and the like.

At certain places, the definitions or embodiments refer to specific rings (e.g., an azetidine ring, a pyridine ring, etc.). Unless otherwise indicated, these rings can be attached to any ring member provided that the valency of the atom is not exceeded.

For example, an azetidine ring may be attached at any position of the ring, whereas a pyridin-3-yl ring is attached at the 3-position.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl, or sulfonyl group.

As used herein, the term “independently selected from” means that each occurrence of a variable or substituent (e.g., each R^{G}), are independently selected at each occurrence from the applicable list.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. C is and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula I, Formula II, etc.) provided herein include stereoisomers of the compounds.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as P-camphorsulfonic acid. Other resolving agents suitable for fractional crystallization methods include stereoisomerically pure forms of a-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide-imidic acid pairs, lactam—lactim pairs, enamine—imine pairs, and annular forms where a proton can occupy two or more positions of a heterocyclic system, for example, 1H- and 3H-imidazole, 1H—, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

Synthesis Compounds of the invention, including salts thereof, can be prepared using known organic synthesis techniques and according to various possible synthetic routes. Example synthetic methods for preparing compounds of the invention are provided in the Schemes below.

Compounds of formula I-16 can be synthesized, for example, using a process shown in Scheme 1. Metal-halogen exchange of I-1 (e.g., Hal¹ and Hal² are independently Cl, Br or I; LG is —NMe₂) followed by reacting with suitable reagents (e.g., DMF, or acetyl chloride) affords compound I-2. Compound I-2 can be converted to compound I-3 with suitable reagent (e.g. sodium borohydride, or, alkyl lithium). The hydroxy group of compound I-3 can be converted to Br to afford compound I-4 under suitable conditions (e.g., PBr₃). Intermediate I-6 is prepared by a process comprising reacting intermediate I-4 with compounds of formula I-5 under strong base condition (e.g., NaHMDS). Compound I-6 can undergo hydrolysis to give compound I-7, which can be converted to intermediate I-8 via the Curtius rearrangement. Compound I-8 can be cyclized to I-9 by treating with suitable reagent (e.g., LDA). Functionalization of lactam group in I-9 with suitable reagent can afford intermediate I-10 (e.g., alkyl halides and base). Intermediate I-12 can be prepared via metal-catalyzed cross coupling reaction of I-10 and suitable reaction with reagent of formula I-11 (e.g., M can be pinacol boronic ester group or tributyltin group). Reduction of intermediate I-12 can afford I-13, which can be further converted into intermediate I-14 using a suitable reagent (e.g., PBr₃). Substitution of I-14 using a suitable nucleophile I-15 can afford compounds of Formula I-16.

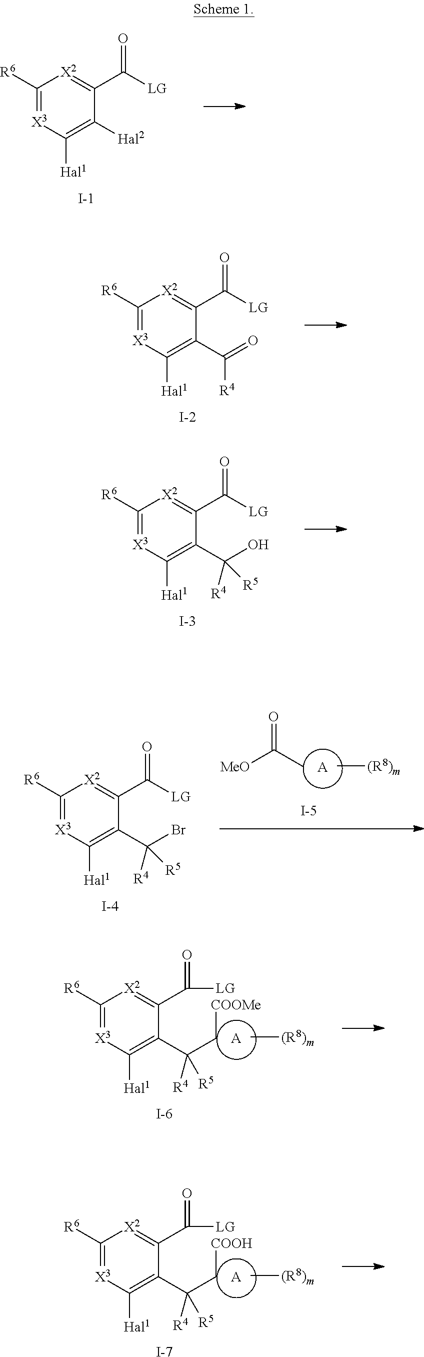

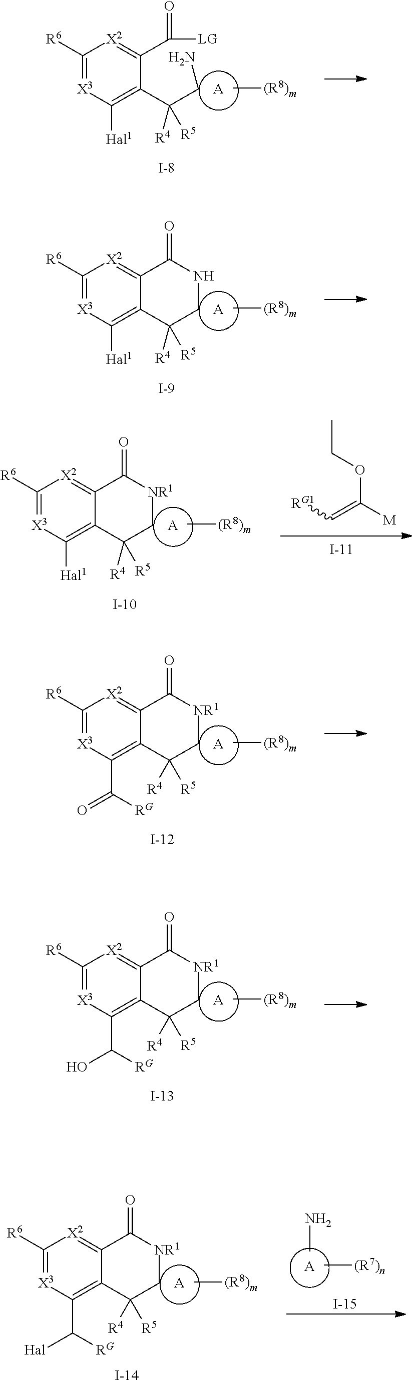

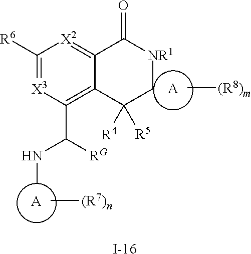

The reactions for preparing compounds of the invention can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

The expressions, “room temperature,” and “r.t.”, as used herein, are understood in the art, and refer generally to a temperature, e.g. a reaction temperature, that is about the temperature of the room in which the reaction is carried out, for example, a temperature from about 20° C. to about 30° C.

### Methods of Use

The present disclosure provides uses for compounds and compositions described herein. The compounds described herein can inhibit the activity of PI3Kα kinase. In some embodiments, provided compounds and compositions are for use in medicine (e.g., as therapy). In some embodiments, provided compounds and compositions are useful in treating a disease, disorder, or condition, wherein an underlying pathology is, wholly or partially, mediated by PI3Kα. In some embodiments, provided compounds and compositions are useful in research as, for example, analytical tools and/or control compounds in biological assays.

In some embodiments, the present disclosure provides methods of administering provided compounds or compositions to a subject in need thereof. In some embodiments, the present disclosure provides methods of administering provided compounds or compositions to a subject suffering from or susceptible to a disease, disorder, or condition associated with PI3Kα. In some embodiments, the present disclosure provides methods of administering provided compounds or compositions to a subject suffering from or susceptible to a disease, disorder, or condition, wherein an underlying pathology is, wholly or partially, mediated by PI3Kα.

In some embodiments, the compounds provided herein are useful as PI3Kα inhibitors. In some embodiments, the present disclosure provides methods of inhibiting PI3Kα in a subject comprising administering a provided compound or composition. In some embodiments, the present disclosure provides methods of inhibiting PI3Kα in a biological sample comprising contacting the sample with a provided compound or composition.

In some embodiments, the present disclosure provides methods of treating a disease, disorder or condition associated with PI3Kα in a subject in need thereof, comprising administering to the subject a compound, salt, or composition of the disclosure. In some embodiments, a disease, disorder or condition is associated with mutation of PI3Kα. In some embodiments, the present disclosure provides methods of treating a disease, disorder or condition, wherein an underlying pathology is, wholly or partially, mediated by PI3Kα, in a subject in need thereof, comprising administering to the subject a provided compound or composition.

In some embodiments, the present disclosure provides methods of treating a variety of PI3Kα-dependent diseases and disorders. In some embodiments, the disease of disorder is a cancer (e.g., breast cancer, brain cancer, prostate cancer, endometrial cancer, gastric cancer, leukemia, lymphoma, sarcoma, colorectal cancer, lung cancer, ovarian cancer, skin cancer, and head and neck cancer). In some embodiments, the disease or disorder associated with PI3Kα includes, but is not limited to, CLOVES syndrome (congenital lipomatous overgrowth, vascular malformations, epidermal naevi, scoliosis/skeletal and spinal syndrome), PIK3CA-related overgrowth syndrome (PROS), endometrial cancer, breast cancer, esophageal squamous-cell cancer, cervical squamous-cell carcinoma, cervical adenocarcinoma, colorectal adenocarcinoma, bladder urothelial carcinoma, glioblastoma, ovarian cancer, non-small-cell lung cancer, esophagogastric cancer, nerve-sheath tumor, head and neck squamous-cell carcinoma, melanoma, esophagogastric adenocarcinoma, soft-tissue sarcoma, prostate cancer, fibrolamellar carcinoma, hepatocellular carcinoma, diffuse glioma, colorectal cancer, pancreatic cancer, cholangiocarcinoma, B-cell lymphoma, mesothelioma, adrenocortical carcinoma, renal non-clear-cell carcinoma, renal clear-cell carcinoma, germ-cell carcinoma, thymic tumor, pheochromocytoma, miscellaneous neuroepithelial tumor, thyroid cancer, leukemia, and encapsulated glioma. In some embodiments, the cancer is breast cancer.

In some embodiments, provided herein is a method of increasing survival or progression-free survival in a patient, comprising administering a compound provided herein to the patient. In some embodiments, the patient has cancer. In some embodiments, the patient has a disease or disorder described herein. As used herein, progression-free survival refers to the length of time during and after the treatment of a solid tumor that a patient lives with the disease but it does not get worse.

Progression-free survival can refer to the length of time from first administering the compound until the earlier of death or progression of the disease. Progression of the disease can be defined by RECIST v. 1.1 (Response Evaluation Criteria in Solid Tumors), as assessed by an independent centralized radiological review committee. In some embodiments, administering of the compound results in a progression free survival that is greater than about 1 month, about 2 months, about 3 months, about 4 months, about 5 months, about 6 months, about 8 months, about 9 months, about 12 months, about 16 months, or about 24 months. In some embodiments, the administering of the compound results in a progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months.

In some embodiments, the administering of the compound results in an increase of progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months.

The present disclosure further provides a compound described herein, or a pharmaceutically acceptable salt thereof, for use in any of the methods described herein.

The present disclosure further provides use of a compound described herein, or a pharmaceutically acceptable salt thereof, for the preparation of a medicament for use in any of the methods described herein.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” a PI3Kα kinase with a compound described herein includes the administration of a compound described herein to an individual or patient, such as a human, having a PI3Kα kinase, as well as, for example, introducing a compound described herein into a sample containing a cellular or purified preparation containing the PI3Kα kinase.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, the term “treating” or “treatment” refers to inhibiting the disease; for example, inhibiting a disease, condition or disorder in an individual who is experiencing or displaying the pathology or symptomatology of the disease, condition or disorder (i.e., arresting further development of the pathology and/or symptomatology) or ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### Combination Therapy

One or more additional therapeutic agents such as, for example, chemotherapeutics or other anti-cancer agents, anti-inflammatory agents, steroids, immunosuppressants, anesthetics (e.g., for use in combination with a surgical procedure), or other agents useful for treating diseases associated with PI3Kα can be used in combination with the compounds and salts provided herein. The agents can be combined with the present compounds in a single dosage form, or the agents can be administered simultaneously or sequentially as separate dosage forms.

Compounds described herein can be used in combination with one or more other kinase inhibitors for the treatment of diseases, such as cancer, that are impacted by multiple signaling pathways. For example, a combination can include one or more inhibitors of the following kinases for the treatment of cancer: Akt1, Akt2, Akt3, TGF-βR, Pim, PKA, PKG, PKC, CaM-kinase, phosphorylase kinase, CDK4/6, MEKK, ERK, MAPK, mTOR, EGFR, HER2, HER3, HER4, INS-R, IGF-1R, IR-R, PDGFαR, PDGFβR, CSFIR, KIT, FLK-II, KDR/FLK-1, FLK-4, fit-1, FGFR1, FGFR2, FGFR3, FGFR4, c-Met, Ron, Sea, TRKA, TRKB, TRKC, FLT3, VEGFR/Flt2, Flt4, EphAl, EphA2, EphA3, EphB2, EphB4, Tie2, Src, Fyn, Lck, Fgr, Btk, Fak, SYK, FRK, JAK, ABL, ALK and B-Raf. Additionally, the solid forms of the inhibitor as described herein can be combined with inhibitors of kinases associated with the PIK3/Akt/mTOR signaling pathway, such as PI3K, Akt (including Akt1, Akt2 and Akt3) and mTOR kinases.

For treating cancer and other proliferative diseases, compounds described herein can be used in combination with targeted therapies, including JAK kinase inhibitors (ruxolitinib, additional JAK½ and JAK1-selective, baricitinib or itacitinib), Pim kinase inhibitors (e.g., LGH447, INCB053914 and SGI-1776), PI3 kinase inhibitors including PI3K-delta selective and broad spectrum PI3K inhibitors (e.g., parsaclisib and INCB50797), PI3K-gamma inhibitors such as PI3K-gamma selective inhibitors, MEK inhibitors, CSF1R inhibitors (e.g., PLX3397 and LY3022855), TAM receptor tyrosine kinases inhibitors (Tyro-3, Axl, and Mer; e.g., INCB81776), angiogenesis inhibitors, interleukin receptor inhibitors, Cyclin Dependent kinase inhibitors (e.g., palbociclib, ribociclib, and abemaciclib), BRAF inhibitors, mTOR inhibitors, proteasome inhibitors (Bortezomib, Carfilzomib), HDAC-inhibitors (panobinostat, vorinostat), DNA methyl transferase inhibitors, dexamethasone, bromo and extra terminal family members inhibitors (for example, bromodomain inhibitors or BET inhibitors, such as OTX015, CPI-0610, INCB54329 or INCB57643), LSD1 inhibitors (e.g., GSK2979552, INCB59872 and INCB60003), estrogen receptor modulators (e.g., fulvestrant), androgen receptor modulators (e.g., enzalutamide), BCL2 inhibitors (e.g., venetoclax), hypoxia-inducible factor-2 alpha inhibitors (e.g., belzutifan), exportin-1 (XPO-1) inhibitors (e.g., selinexor), KRAS inhibitors (e.g., sotorasib), arginase inhibitors (e.g., INCB1158), indoleamine 2,3-dioxygenase inhibitors (e.g., epacadostat, NLG919 or BMS-986205), PARP inhibiors (e.g., olaparib or rucaparib), and inhibitors of BTK such as ibrutinib.

For treating cancer and other proliferative diseases, compounds described herein can be used in combination with chemotherapeutic agents, agonists or antagonists of nuclear receptors, or other anti-proliferative agents. Compounds described herein can also be used in combination with a medical therapy such as surgery or radiotherapy, e.g., gamma-radiation, neutron beam radiotherapy, electron beam radiotherapy, proton therapy, brachytherapy, and systemic radioactive isotopes.

Examples of suitable chemotherapeutic agents include any of: abarelix, abiraterone, afatinib, aflibercept, aldesleukin, alemtuzumab, alitretinoin, allopurinol, altretamine, amidox, amsacrine, anastrozole, aphidicolon, arsenic trioxide, asparaginase, axitinib, azacitidine, bevacizumab, bexarotene, baricitinib, bendamustine, bicalutamide, bleomycin, bortezombi, bortezomib, brivanib, buparlisib, busulfan intravenous, busulfan oral, calusterone, camptosar, capecitabine, carboplatin, carmustine, cediranib, cetuximab, chlorambucil, cisplatin, cladribine, clofarabine, crizotinib, cyclophosphamide, cytarabine, dacarbazine, dacomitinib, dactinomycin, dalteparin sodium, dasatinib, dactinomycin, daunorubicin, decitabine, degarelix, denileukin, denileukin diftitox, deoxycoformycin, dexrazoxane, didox, docetaxel, doxorubicin, droloxafine, dromostanolone propionate, eculizumab, enzalutamide, epidophyllotoxin, epirubicin, epothilones, erlotinib, estramustine, etoposide phosphate, etoposide, exemestane, fentanyl citrate, filgrastim, floxuridine, fludarabine, fluorouracil, flutamide, fulvestrant, gefitinib, gemcitabine, gemtuzumab ozogamicin, goserelin acetate, histrelin acetate, ibritumomab tiuxetan, idarubicin, idelalisib, ifosfamide, imatinib mesylate, interferon alfa 2a, irinotecan, lapatinib ditosylate, lenalidomide, letrozole, leucovorin, leuprolide acetate, levamisole, lonafarnib, lomustine, meclorethamine, megestrol acetate, melphalan, mercaptopurine, methotrexate, methoxsalen, mithramycin, mitomycin C, mitotane, mitoxantrone, nandrolone phenpropionate, navelbene, necitumumab, nelarabine, neratinib, nilotinib, nilutamide, niraparib, nofetumomab, oserelin, oxaliplatin, paclitaxel, pamidronate, panitumumab, panobinostat, pazopanib, pegaspargase, pegfilgrastim, pemetrexed disodium, pentostatin, pilaralisib, pipobroman, plicamycin, ponatinib, porfimer, prednisone, procarbazine, quinacrine, ranibizumab, rasburicase, regorafenib, reloxafine, revlimid, rituximab, rucaparib, ruxolitinib, sorafenib, streptozocin, sunitinib, sunitinib maleate, tamoxifen, tegafur, temozolomide, teniposide, testolactone, tezacitabine, thalidomide, thioguanine, thiotepa, tipifarnib, topotecan, toremifene, tositumomab, trastuzumab, tretinoin, triapine, trimidox, triptorelin, uracil mustard, valrubicin, vandetanib, vinblastine, vincristine, vindesine, vinorelbine, vorinostat, veliparib, talazoparib, and zoledronate.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

Example anti-inflammatory agents include, but are not limited to, aspirin, choline salicylates, celecoxib, diclofenac potassium, diclofenac sodium, diclofenac sodium with misoprostol, diflunisal, etodolac, fenoprofen, flurbiprofen, ibuprofen, ketoprofen, meclofenamate sodium, mefenamic acid, nabumetone, naproxen, naproxen sodium, oxaprozin, piroxican, rofecoxib, salsalate, sodium salicylate, sulindac, tolmetin sodium, and valdecoxib.

Example steroids include, but are not limited to, corticosteroids such as cortisone, dexamethasone, hydrocortisone, methylprednisolone, prednisolone, and prednisone.

Example immunosuppressants include, but are not limited to, azathioprine, chlorambucil, cyclophosphamide, cyclosporine, daclizumab, infliximab, methotrexate, and tacrolimus.

Example anesthetics include, but are not limited, to local anesthetics (e.g., lidocaine, procain, ropivacaine) and general anesthetics (e.g., desflurane, enflurane, halothane, isoflurane, methoxyflurane, nitrous oxide, sevoflurane, mmobarbital, methohexital, thiamylal, thiopental, diazepam, lorazepam, midazolam, etomidate, ketamine, propofol, alfentanil, fentanyl, remifentanil, buprenorphine, butorphanol, hydromorphone levorphanol, meperidine, methadone, morphine, nalbuphine, oxymorphone, pentazocine).

In some embodiments, the additional therapeutic agent is administered simultaneously with a compound or salt provided herein. In some embodiments, the additional therapeutic agent is administered after administration of the compound or salt provided herein. In some embodiments, the additional therapeutic agent is administered prior to administration of the compound or salt provided herein. In some embodiments, the compound or salt provided herein is administered during a surgical procedure. In some embodiments, the compound or salt provided herein is administered in combination with an additional therapeutic agent during a surgical procedure.

As provided herein, the additional compounds, inhibitors, agents, etc. can be combined with the compounds provided herein in a single or continuous dosage form, or they can be administered simultaneously or sequentially as separate dosage forms.

### Pharmaceutical Formulations and Dosage Forms

When employed as pharmaceuticals, the compounds of the invention can be administered in the form of pharmaceutical compositions which refers to a combination of a compound of the invention, or its pharmaceutically acceptable salt, and at least one pharmaceutically acceptable carrier. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal, or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, one or more of the compounds of the invention above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid pre-formulation composition containing a homogeneous mixture of a compound of the present invention. When referring to these pre-formulation compositions as homogeneous, the active ingredient is typically dispersed evenly throughout the composition so that the composition can be readily subdivided into equally effective unit dosage forms such as tablets, pills and capsules. This solid pre-formulation is then subdivided into unit dosage forms of the type described above.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

The liquid forms in which the compounds and compositions of the present invention can be incorporated for administration orally or by injection include aqueous solutions, suitably flavored syrups, aqueous or oil suspensions, and flavored emulsions with edible oils.

The compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the disclosure can further include one or more additional pharmaceutical agents such as a chemotherapeutic, steroid, anti-inflammatory compound, or immunosuppressant, examples of which are provided herein.

### Labeled Compounds and Assay Methods

Another aspect of the present invention relates to fluorescent dye, spin label, heavy metal or radio-labeled compounds of the invention that would be useful not only in imaging but also in assays, both in vitro and in vivo, for localizing and quantitating the PI3Kα enzyme in tissue samples, including human, and for identifying PI3Kα enzyme ligands by inhibition binding of a labeled compound. Accordingly, the present invention includes PI3Kα enzyme assays that contain such labeled compounds.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro FGFR enzyme labeling and competition assays, compounds that incorporate ³H, ¹⁴C, ⁸²Br, ¹²⁵I, ¹³¹I, or ³⁵S will generally be most useful. For radio-imaging applications ¹¹C ¹⁸F, ¹²⁵I, ¹²³I, ¹²⁴I, ¹³¹I, ⁷⁵Br, ⁷⁶Br or ⁷⁷Br will generally be most useful.

One or more constituent atoms of the compounds presented herein can be replaced or substituted with isotopes of the atoms in natural or non-natural abundance. In some embodiments, one or more atoms are replaced or substituted by deuterium. For example, one or more hydrogen atoms in a compound of the present disclosure can be replaced by deuterium atoms (e.g., one or more hydrogen atoms of a C₁₋₆ alkyl group of Formula I can be optionally substituted with deuterium atoms, such as —CD₃ being substituted for —CH₃). In some embodiments, alkyl groups of the disclosed Formulas (e.g., the compound of any of Formulas I-VII) can be perdeuterated.

In some embodiments, the compound provided herein (e.g., the compound of any of Formulas I-VII), or a pharmaceutically acceptable salt thereof, comprises at least one deuterium atom.

In some embodiments, the compound provided herein (e.g., the compound of any of Formulas I-VII), or a pharmaceutically acceptable salt thereof, comprises two or more deuterium atoms.

In some embodiments, the compound provided herein (e.g., the compound of any of Formulas I-VII), or a pharmaceutically acceptable salt thereof, comprises three or more deuterium atoms.

In some embodiments, for a compound provided herein (e.g., the compound of any of Formulas I-VII), or a pharmaceutically acceptable salt thereof, all of the hydrogen atoms are replaced by deuterium atoms (i.e., the compound is “perdeuterated”).

It is understood that a “radio-labeled “or “labeled compound” is a compound that has incorporated at least one radionuclide. In some embodiments the radionuclide is selected from the group consisting of ³H, ¹⁴C, ¹²⁵I, ³⁵S and ⁸²Br.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

A radio-labeled compound of the invention can be used in a screening assay to identify/evaluate compounds. In general terms, a newly synthesized or identified compound (i.e., test compound) can be evaluated for its ability to reduce binding of the radio-labeled compound of the invention to the PI3Kα enzyme. Accordingly, the ability of a test compound to compete with the radio-labeled compound for binding to the PI3Kα enzyme directly correlates to its binding affinity.

### Kits

The present invention also includes pharmaceutical kits useful, for example, in the treatment or prevention of PI3Kα-associated diseases or disorders referred to herein which include one or more containers containing a pharmaceutical composition comprising a therapeutically effective amount of a compound of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples were found to be inhibitors of PI3Kα as described below.

### Examples

Experimental procedures for compounds of the invention are provided below. Preparatory LC-MS purifications of some of the compounds prepared were performed on Waters mass directed fractionation systems. The basic equipment setup, protocols, and control software for the operation of these systems have been described in detail in the literature. See e.g. “Two-Pump At Column Dilution Configuration for Preparative LC-MS”, K. Blom, J. Combi. Chem., 4, 295 (2002); “Optimizing Preparative LC-MS Configurations and Methods for Parallel Synthesis Purification”, K. Blom, R. Sparks, J. Doughty, G. Everlof, T. Haque, A. Combs, J. Combi. Chem., 5, 670 (2003); and “Preparative LC-MS Purification: Improved Compound Specific Method Optimization”, K. Blom, B. Glass, R. Sparks, A. Combs, J. Combi. Chem., 6, 874-883 (2004). The compounds separated were typically subjected to analytical liquid chromatography mass spectrometry (LCMS) for purity check.

Some of the compounds prepared were also separated on a preparative scale by reverse-phase high performance liquid chromatography (RP-HPLC) with MS detector or flash chromatography (silica gel) as indicated in the Examples.

The following abbreviations may be used herein: DCM (dichlorometlane); DMF (N, N--dimethylformamide); g (grarm(s)); h (hour(s)); HCl (hydrochloric acid); HPLC (high performance liquid chromatography); LCMS (liquid chromatography-mass spectronetry LDA (lithium diisopropylarmide); M (rmolar); NIS (Mass spectrometry); MeOH (methanol); mg (milligram(s)); min. (minutes(s)); mL (milliliter(s)) mmol (midlimoMe(s)); nM (nanomolar); RP-HPLC (reverse phase high performance liquid chromatography); r.t. (or “rt”) (room temperature), TFA (trifluoroacetic acid); THF (tetrahydrofuran); μg (microgram(s)); μl (microliter(s)); μM (micromolar). Brine is saturated aqueous sodium chloride. In vacuo is under vacuum.

### Example 1. 2-[1-(2,7-Dimethyl-1-oxo-spiro[4H-isoquinoline-3,1′-indane]-5yl)ethylamino]benzoic acid

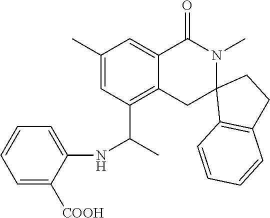 [SMILES: Cc1cc2c(c(C(C)Nc3ccccc3C(=O)O)c1)CC1(CCc3ccccc31)N(C)C2=O]

### Step 1: 3-Bromo-2-formyl-N,N,5-trimethyl-benzamide

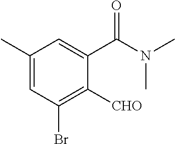

To a mixture of 3-bromo-2-iodo-N,N,5-trimethyl-benzamide (2.60 g, 7.07 mmol) in 60 mL anhydrous THE at −40° C. was added isopropylmagnesium chloride lithium chloride solution (8.15 mL, 10.60 mmol). The resulting mixture was stirred at −40° C. for 30 min, followed by addition of N,N-dimethylformamide (2.20 mL, 28.26 mol) dropwise at the same temperature. The resulting mixture was warmed to rt and diluted with water (100 mL) and extracted with ethyl acetate (3×50 mL). The combined organic layers were washed with brine (50 mL), dried over anhydrous sodium sulfate, and filtered. After filtration, the filtrate was concentrated under reduced pressure. The resulting residue was purified by silica gel column chromatography and eluted with ethyl acetate and hexane (0 to 100%) to afford the desired product as a pale-yellow solid. LCMS calculated for C₁₁H₁₃BrNO₂ (M+H)⁺m/z=272.0; found 272.0.

### Step 2: 3-Bromo-2-(hydroxymethyl)-N,N,5-trimethyl-benzamide

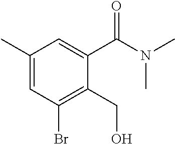

To a solution of 3-bromo-2-formyl-N,N,5-trimethyl-benzamide (200.0 mg, 0.74 mmol) in a mixture of 4 mL DCM and 4 mL MeOH at 0° C. was added sodium borohydride (28.0 mg, 0.74 mmol) in three portions. The mixture was stirred at the same temperature for 1 h. The reaction mixture was then poured into a mixture of 10 mL 0.5 N HCl and 10 mL DCM. The aqueous layer was extracted with 10 mL DCM three times. The combined organic layers were washed with brine (20 mL), dried over anhydrous sodium sulfate, and filtered. After filtration, the filtrate was concentrated under reduced pressure. The resulting residue was purified by silica gel column chromatography and eluted with ethyl acetate and DCM (0 to 100%) to afford the desired product as a white solid. LCMS calculated for C₁₁H₁₅BrNO₂ (M+H)⁺m/z=274.0; found 274.0.

### Step 3: 3-Bromo-2-(bromomethyl)-N,N,5-trimethyl-benzamide

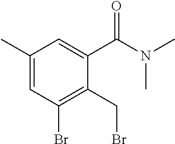

To a solution of 3-bromo-2-(hydroxymethyl)-N,N,5-trimethyl-benzamide (145.0 mg, 0.53 mmol) in 5 mL DCM at 0° C. was added a solution of phosphorus tribromide (101.2 μL, 1.07 mmol) in 2 mL DCM dropwise. The resulting mixture was stirred at the same temperature for 1 h. The reaction mixture was then diluted with 10 mL water and extracted with DCM (3×10 mL). The combined organic layers were washed with brine (20 mL), dried over anhydrous sodium sulfate, and filtered. After filtration, the filtrate was concentrated under reduced pressure. The resulting residue was purified by silica gel column chromatography and eluted with ethyl acetate in DCM (0 to 80%) to afford the desired product as a white solid. LCMS calculated for C₁₁H₁₄Br₂NO (M+H)⁺m/z=335.9; found 335.8.

### Step 4: Methyl 1-[[2-bromo-6-(dimethylcarbamoyl)-4-methyl-phenyl]methyl]indane-1-carboxylate

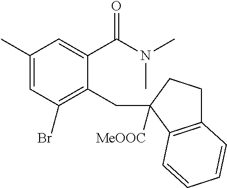 [SMILES: COC(=O)C1(Cc2c(Br)cc(C)cc2C(=O)N(C)C)CCc2ccccc21]

To a solution of methyl indane-1-carboxylate (437.6 mg, 2.48 mmol) in 15 mL anhydrous THE at −78° C. was added [bis(trimethylsilyl)amino]sodium (2.48 mL, 2.48 mmol) dropwise. The resulting solution was stirred at the same temperature for 30 min, then a solution of 3-bromo-2-(bromomethyl)-N,N,5-trimethyl-benzamide (520.0 mg, 1.55 mmol) in 5 mL anhydrous THE was added dropwise. The reaction mixture was stirred at −78° C. for another 1 hour, then poured into a mixture of 50 mL saturated ammonium chloride and 50 mL DCM. The aqueous layer was extracted with DCM (3×50 mL). The combined organic layers were washed with brine (50 mL), dried over anhydrous sodium sulfate, and filtered. After filtration, the filtrate was concentrated under reduced pressure. The resulting residue was purified by silica gel column chromatography, eluted with ethyl acetate and DCM (0 to 80%) to afford the desired product. LCMS calculated for C₂₂H₂₅BrNO₃ (M+H)⁺m/z=432.1; found 432.1.

### Step 5: 1-[[2-Bromo-6-(dimethylcarbamoyl)-4-methyl-phenyl]methyl]indane-1-carboxylic acid

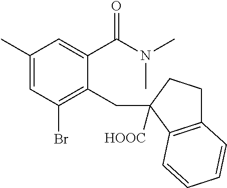 [SMILES: Cc1cc(Br)c(CC2(C(=O)O)CCc3ccccc32)c(C(=O)N(C)C)c1]

To a solution of methyl 1-[[2-bromo-6-(dimethylcarbamoyl)-4-methyl-phenyl]methyl]indane-1-carboxylate (34.0 mg, 0.08 mmol) in a mixture of 1 mL THE and 0.5 mL MeOH at 0° C. was added 0.25 mL 2N NaOH solution. The mixture was heated to 50° C. and stirred for 5 h. Upon cooling to room temperature, the reaction mixture was diluted with 1 mL 1N HCl solution, 10 mL water, and extracted with DCM (3×10 mL). The combined organic layers were washed with brine (20 mL), dried over anhydrous sodium sulfate, and filtered. After filtration, the filtrate was concentrated under reduced pressure to give the crude product, which was directly used in the following step without further purification. LCMS calculated for C₂₁H₂₃BrNO₃ (M+H)⁺m/z=418.1; found 418.1.

### Step 6: 2-[(1-Aminoindan-1-yl)methyl]-3-bromo-N,N,5-trimethyl-benzamide

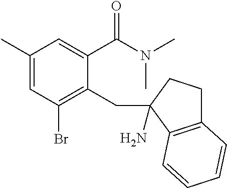 [SMILES: Cc1cc(Br)c(CC2(N)CCc3ccccc32)c(C(=O)N(C)C)c1]

To a solution of 1-[[2-bromo-6-(dimethylcarbamoyl)-4-methyl-phenyl]methyl]indane-1-carboxylic acid (486.0 mg, 1.17 mmol) in 12 mL toluene was added N,N-diethylethanamine (196.9 μL, 1.40 mmol) and [azido(phenoxy)phosphoryl]oxybenzene (276.1 μL, 1.28 mmol). The mixture was vacuumed and backfilled with nitrogen gas three times. The reaction mixture was then heated to 100° C. for 5 h. The mixture was then cooled down to 0° C. and sodium trimethylsilanolate (261.9 mg, 2.33 mmol) in 2.3 mL THE was added. Then the mixture was warmed to r.t. and stirred for 30 min. After quenching the reaction with 5% citric acid (20 mL), the pH of the mixture was adjusted to 12 by adding 2N NaOH solution. The mixture was extracted with DCM (3×20 mL). The combined organic layers were washed with brine (20 mL), dried over anhydrous sodium sulfate, and filtered. After filtration, the filtrate was concentrated under reduced pressure. The resulting residue was purified by silica gel column chromatography, eluted with ethyl acetate in dichloromethane (0 to 100%), then methanol in dichloromethane (0 to 20%) to afford the desired product. LCMS calculated for C₂₀H₂₄BrN₂O (M+H)⁺m/z=389.1; found 389.1.

### Step 7: 5-Bromo-7-methyl-spiro[2,4-dihydroisoquinoline-3,1′-indane]-]-one

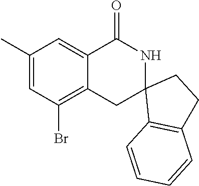 [SMILES: Cc1cc(Br)c2c(c1)C(=O)NC1(CCc3ccccc31)C2]

To a solution of 2-[(1-aminoindan-1-yl)methyl]-3-bromo-N,N,5-trimethyl-benzamide (362.0 mg, 0.93 mmol) in 10 mL anhydrous THE at −78° C. was added 2M LDA (0.70 mL, 1.40 mmol) dropwise. The resulting solution was stirred at the same temperature for 15 min, then poured into the mixture of 10 mL saturated ammonium chloride and 10 mL DCM. The aqueous layer was extracted with DCM (3×10 mL). The combined organic layers were washed with brine (20 mL), dried over anhydrous sodium sulfate, and filtered. After filtration, the filtrate was concentrated under reduced pressure. The resulting residue was purified by silica gel column chromatography, eluted with MeOH in DCM (0 to 20%) to afford the desired product as a white solid. LCMS calculated for C₁₈H₁₇BrNO (M+H)⁺m/z=344.1; found 344.0.

### Step 8: 5-Bromo-2,7-dimethyl-spiro[4H-isoquinoline-3,1′-indane]-I-one

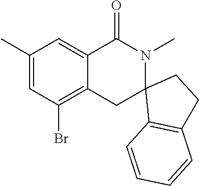 [SMILES: Cc1cc(Br)c2c(c1)C(=O)N(C)C1(CCc3ccccc31)C2]

To a solution of 5-bromo-7-methyl-spiro[2,4-dihydroisoquinoline-3,1′-indane]-1-one, (160.0 mg, 0.47 mmol) in 5 mL anhydrous DMF at 0° C. was added sodium hydride (28.1 mg, 0.70 mmol). The resulting solution was stirred at the same temperature for 30 min, then iodomethane (45.3 μL, 0.70 mmol) was added dropwise. The mixture was slowly warmed to room temperature and stirred for another 1 h. Then the reaction solution was poured into a mixture of 10 mL saturated ammonium chloride and 10 mL ethyl acetate. The aqueous layer was extracted with ethyl acetate (3×10 mL). The combined organic layers were washed with brine (20 mL), dried over anhydrous sodium sulfate, and filtered. After filtration, the filtrate was concentrated under reduced pressure. The resulting residue was purified by silica gel column chromatography, eluted with ethyl acetate in dichloromethane (0 to 100%) to afford the desired product. LCMS calculated for Cl₉H₁₉BrNO (M+H)⁺m/z=358.1; found 358.0.

### Step 9: 5-Acetyl-2,7-dimethyl-spiro[4H-isoquinoline-3,1′-indane]-I-one

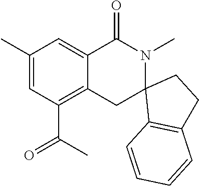 [SMILES: CC(=O)c1cc(C)cc2c1CC1(CCc3ccccc31)N(C)C2=O]

A mixture of 5-bromo-2,7-dimethyl-spiro[4H-isoquinoline-3,1′-indane]-1-one (100.0 mg, 0.28 mmol), [1,1′-Bis(diphenylphosphino)ferrocene]dichloropalladium(II) (19.7 mg, 0.03 mmol), and tributyl(1-ethoxyvinyl)stannane (202.8 mg, 0.56 mmol) in dioxane (3 mL) was heated at 95° C. for 12 h under nitrogen atmosphere. Upon cooling to room temperature, 1 mL 1 N HCl was added to the reaction mixture and stirred for 30 min. The mixture was diluted with 10 mL water and extracted with ethyl acetate (3×10 mL). The combined organic layers were washed with brine (20 mL), dried over anhydrous sodium sulfate, and filtered. After filtration, the filtrate was concentrated under reduced pressure. The resulting residue was purified by silica gel column chromatography, eluted with ethyl acetate in dichloromethane (0 to 100%) to afford the desired product. LCMS calculated for: C₂₁H₂₂NO₂ (M+H)⁺m/z=320.2; found 320.1.

### Step 10: 5-(1-Hydroxyethyl)-2, 7-dimethyl-spiro[4H-isoquinoline-3, 1′-indane]-I-one

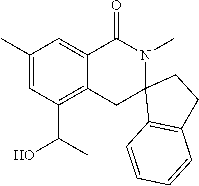 [SMILES: Cc1cc2c(c(C(C)O)c1)CC1(CCc3ccccc31)N(C)C2=O]

To a solution of 5-acetyl-2,7-dimethyl-spiro[4H-isoquinoline-3,1′-indane]-1-one (76.0 mg, 0.24 mmol) in a mixture of 2 mL DCM and 2 mL MeOH at 0° C. was added sodium borohydride (13.5 mg, 0.36 mmol). The mixture was stirred at the same temperature for 1 h. The reaction mixture was then poured into a mixture of 10 mL 0.5 N HCl and 10 mL DCM. The aqueous layer was extracted with 10 mL DCM three times. The combined organic layers were washed with brine (20 mL), dried over anhydrous sodium sulfate, and filtered. After filtration, the filtrate was concentrated under reduced pressure. The resulting residue was purified by silica gel column chromatography, eluted with ethyl acetate and DCM (0 to 100%) to afford the desired product. LCMS calculated for C₂₁H₂₄NO₂(M+H)⁺m/z=322.2; found 322.1.

### Step 11: 5-(1-Bromoethyl)-2, 7-dimethyl-spiro[4H-isoquinoline-3,1′-indane]-I-one

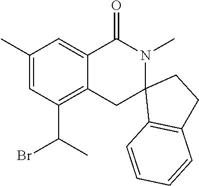 [SMILES: Cc1cc2c(c(C(C)Br)c1)CC1(CCc3ccccc31)N(C)C2=O]

To a solution of 5-(1-hydroxyethyl)-2,7-dimethyl-spiro[4H-isoquinoline-3,1′-indane]-1-one (98.0 mg, 0.30 mmol) in 3 mL DCM at 0° C. was added a solution of phosphorus tribromide (101.2 μL, 1.07 mmol) in 1 mL DCM dropwise. The resulting mixture was stirred at the same temperature for 1 h. The reaction mixture was then diluted with 10 mL water and extracted with DCM (3×10 mL). The combined organic layers were washed with brine (20 mL), dried over anhydrous sodium sulfate, and filtered. After filtration, the filtrate was concentrated under reduced pressure. The resulting residue was purified by silica gel column chromatography, eluted with ethyl acetate in DCM (0 to 100%) to afford the desired product as a white solid. LCMS calculated for C₂₁H₂₃BrNO (M+H)⁺m/z=386.1; found 386.1.

### Step 12: 2-[ ]-(2,7-Dimethyl-1-oxo-spiro[4H-isoquinoline-3,1′-indane]-5-yl)ethylamino]benzoic acid

To a solution of 5-(1-bromoethyl)-2,7-dimethyl-spiro[4H-isoquinoline-3,1′-indane]-1-one (20.0 mg, 0.05 mmol) in 0.5 mL DMF was added 2-aminobenzoic acid (21.4 mg, 0.16 mmol). The resulting mixture was heated to 80° C. for 2 h. Upon cooling to room temperature, the mixture was diluted with methanol and purified by prep-HPLC (column: Sunfire prep C18 column, 30*150 mm, 5 m; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₈H₂₉N₂O₃ (M+H)⁺m/z=441.2; found 441.2.

### Example 2. 3-[1-(2,7-Dimethyl-1-oxo-spiro[4H-isoquinoline-3,1′-indane]-5-yl)ethylamino]-6-methyl-pyridine-2-carboxylic acid

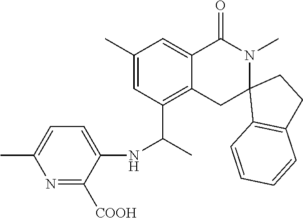 [SMILES: Cc1cc2c(c(C(C)Nc3ccc(C)nc3C(=O)O)c1)CC1(CCc3ccccc31)N(C)C2=O]

The title compound was prepared using similar procedures as described for Example 1, with 3-amino-6-methylpicolinic acid replacing 2-aminobenzoic acid in Step 12. LCMS calculated for C₂₈H₃₀N₃O₃ (M+H)⁺m/z=456.2; found 456.2.

### Example 3. 6-Chloro-3-[1-(2,7-dimethyl-1-oxo-spiro[4H-isoquinoline-3,1′-indane]-5-yl)ethylamino]pyridine-2-carboxylic acid

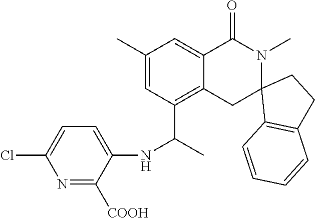 [SMILES: Cc1cc2c(c(C(C)Nc3ccc(Cl)nc3C(=O)O)c1)CC1(CCc3ccccc31)N(C)C2=O]

The title compound was prepared using similar procedures as described for Example 1, with 3-amino-6-chloropicolinic acid replacing 2-aminobenzoic acid in Step 12. LCMS calculated for C₂₇H₂₇ClN₃O₃(M+H)⁺m/z=476.2; found 476.1.

### Example 4. 2-[1-(2,7-Dimethyl-1-oxo-spiro[4H-isoquinoline-3,2′-indane]-5-yl)ethylamino]benzoic acid

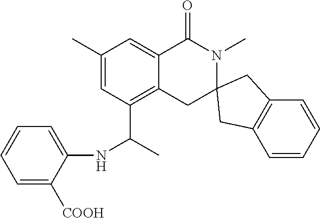 [SMILES: Cc1cc2c(c(C(C)Nc3ccccc3C(=O)O)c1)CC1(Cc3ccccc3C1)N(C)C2=O]

The title compound was prepared using similar procedures as described for Example 1, with methyl 2,3-dihydro-1H-indene-2-carboxylate replacing methyl 2,3-dihydro-1H-indene-1-carboxylate in Step 4. LCMS calculated for C₂₈H₂₉N₂O₃ (M+H)⁺m/z=441.2; found 441.4.

### Example 5. 3-((1-(2′,7′-Dimethyl-1′-oxo-1,1′,3,4′-tetrahydro-2′H-spiro[indene-2,3′-isoquinolin]-5′-yl)ethyl)amino)-6-methylpicolinic acid

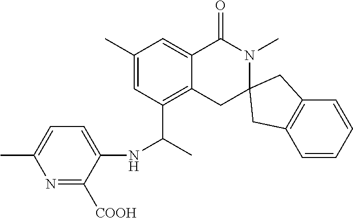 [SMILES: Cc1cc2c(c(C(C)Nc3ccc(C)nc3C(=O)O)c1)CC1(Cc3ccccc3C1)N(C)C2=O]

The title compound was prepared using similar procedures as described for Example 1, with methyl 2,3-dihydro-1H-indene-2-carboxylate replacing methyl 2,3-dihydro-1H-indene-1-carboxylate in Step 4, and 3-amino-6-methylpicolinic acid replacing 2-aminobenzoic acid in Step 12. LCMS calculated for C₂₈H₃₀N₃O₃ (M+H)⁺m/z=456.2; found 456.4.

### Example 6. 5-[1-[[6-Chloro-2-(1-methylpyrazol-4-yl)-3-pyridyl]amino]ethyl]-2,7-dimethyl-spiro[4H-isoquinoline-3,1′-indane]-1-one

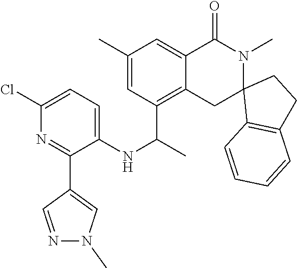 [SMILES: Cc1cc2c(c(C(C)Nc3ccc(Cl)nc3-c3cnn(C)c3)c1)CC1(CCc3ccccc31)N(C)C2=O]

### Step 1: 6-Chloro-2-(1-methylpyrazol-4-yl)pyridin-3-amine

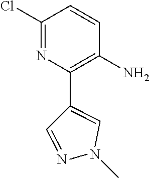

To a solution of 2-bromo-6-chloro-pyri in-3-amine (500.0 mg, 2.41 mmol) in a mixture of 8 mL dioxane and 2 mL water was added tetrakis(triphenylphosphine)palladium(0) (557.0 mg, 0.48 mmol), followed by 1-methyl-4-(4,4,5,5-tetramethyl-1,3,2-dioxaborolan-2-yl)pyrazole (501.5 mg, 2.41 mmol) and cesium carbonate (2355.8 mg, 7.23 mmol). The mixture was vacuumed and backfilled with nitrogen gas three times. The reaction was then heated to 80° C., stirred for 1 h, then poured into a mixture of 50 mL DCM and 50 mL water. The aqueous layer was extracted with DCM (3×20 mL). The combined organic layers were washed with brine (50 mL), dried over anhydrous sodium sulfate, and filtered. After filtration, the filtrate was concentrated under reduced pressure. The resulting residue was purified by silica gel column chromatography and eluted with ethyl acetate and DCM (0 to 100%) to afford the desired product. LCMS calculated for C₉H₁₀ClN₄ (M+H)⁺m/z=209.1 found 209.0.

### Step 2: 5-[1-[[6-Chloro-2-(1-methylpyrazol-4-yl)-3-pyridyl]amino]ethyl]-2, 7-dimethyl-spiro[4H-isoquinoline-3,1′-indane]-]-one

The title compound was prepared using similar procedures as described for Example 1, with 6-chloro-2-(1-methylpyrazol-4-yl)pyridin-3-amine replacing 2-aminobenzoic acid in Step 12. LCMS calculated for C₃₀H₃₁ClN₅O (M+H)⁺m/z=512.2; found 512.1.

### Example 7. 5-[1-[[6-Chloro-2-(3-fluoro-4-pyridyl)-3-pyridyl]amino]ethyl]-2,7-dimethyl-spiro[4H-isoquinoline-3,1′-indane]-1-one

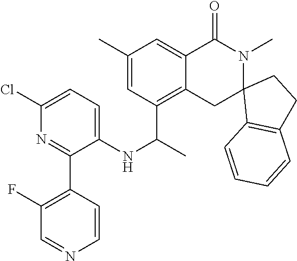 [SMILES: Cc1cc2c(c(C(C)Nc3ccc(Cl)nc3-c3ccncc3F)c1)CC1(CCc3ccccc31)N(C)C2=O]

### Step 1. 6-Chloro-2-(3-fluoro-4-pyridyl)pyridin-3-amine

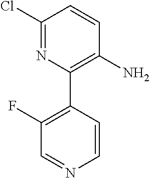 [SMILES: Nc1ccc(Cl)nc1-c1ccncc1F]

To a solution of 2-bromo-6-chloro-pyridin-3-amine (500.0 mg, 2.41 mmol) in a mixture of 8 mL toluene and 2 mL water was added tetrakis(triphenylphosphine)palladium(0) (557.0 mg, 0.48 mmol) followed by 3-fluoro-4-(4,4,5,5-tetramethyl-1,3,2-dioxaborolan-2-yl)pyridine (537.6 mg, 2.41 mmol) and cesium carbonate (2355.8 mg, 7.23 mmol). The mixture was vacuumed and backfilled with nitrogen gas three times. The reaction was then heated to 80° C., stirred for 3 h, then poured into a mixture of 50 mL DCM and 50 mL water. The aqueous layer was extracted with DCM (3×20 mL). The combined organic layers were washed with brine (50 mL), dried over anhydrous sodium sulfate, and filtered.

After filtration, the filtrate was concentrated under reduced pressure. The resulting residue was purified by silica gel column chromatography and eluted with ethyl acetate and DCM (0 to 100%) to afford the desired product. LCMS calculated for C₁₀H₈ClFN₃ (M+H)⁺m/z=224.0 found 224.1.

### Step 2. 5-[1-[[6-Chloro-2-(3-fluoro-4-pyridyl)-3-pyridyl]amino]ethyl]-2, 7-dimethyl-spiro[4H-isoquinoline-3,1′-indane]-]-one

The title compound was prepared using similar procedures as described for Example 1, with 6-chloro-2-(3-fluoro-4-pyridyl)pyridin-3-amine replacing 2-aminobenzoic acid in Step 12. LCMS calculated for C₃₁H₂₉ClFN₄O (M+H)⁺m/z=527.2; found 527.1.

### Example A. pS473 AKT Assay Protocol

T47D (PIK3CA H1047R) cells were cultured using 10-cm petri dishes with recommended medium plus 10% fetal bovine serum. One day before the assay, cells were seeded in 96-well plates. After overnight incubation, the medium was changed to DMEM with 2.5% fetal bovine serum (FBS) and the cells were treated with different concentrations of the compounds for 2 h. Cells were then fixed using 4% paraformaldehyde at room temperature for 20 min. Aspirate 4% paraformaldehyde, and wash cells using 1× regular phosphate buffered saline 3 times, 5 min each. Aspirate any residual phosphate buffered saline and block cells using 10% goat serum containing 1% bovine serum albumin and 0.3% Triton X-100 at room temperature for 1 h. Without any additional washing, primary antibodies (rabbit anti-pSer473 AKT) were diluted using blocking buffer and added at a final volume of 50 microliter per well. Keep assay plates with primary antibodies overnight at 4° C. Wash cells using 1× regular phosphate buffered saline 3 times, 5 min each. After the final wash, incubate cells with horseradish peroxidase-conjugated secondary antibodies (goat Anti-rabbit IgG) diluted using the same blocking buffer at room temperature for 1 h. Wash cells thoroughly using 1× regular phosphate buffered saline 3 times, 5 min each. Aspirate any residual phosphate buffered saline. Add Super-Signal ELISA Pico Chemiluminescent Substrate at a final volume of 100 microliter per well. Read plates on i3× Multi-Mode Microplate Reader and calculate IC₅₀ values using GraphPad Prism software.

Results of the assay described above are presented in Table 1. “+” indicates an IC₅₀ less than 1000 nM; “++” indicates an IC₅₀ greater than or equal to 1000 nM but less than 5000 nM; “+++” indicates an IC₅₀ greater than or equal to 5000 nM but less than 10000 nM; and “++++” indicates an IC₅₀ greater than or equal to 10000 nM.

TABLE 1

| Example | T47D_IC₅₀ (nM) |
|---|---|
| 1 | + |
| 2 | + |
| 3 | + |
| 4 | ++ |
| 5 | ++ |
| 6 | + |
| 7 | + |

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound of Formula I: 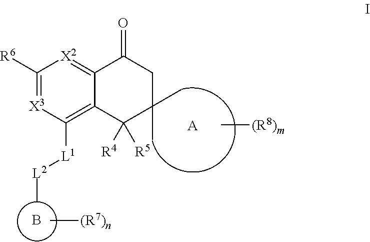
or a pharmaceutically acceptable salt thereof, wherein:
X¹ is NR¹ or O;
X² is CR² or N;
X³ is CR³ or N;
n is 0, 1, 2, 3, 4, 5, or 6;
m is 0, 1, 2, 3, 4, 5, or 6;
Ring A is C₃₋₁₄ cycloalkyl or 4-14 membered heterocycloalkyl;
Ring B is C₃₋₁₀ cycloalkyl, C₆₋₁₀ aryl, 4-10 membered heterocycloalkyl, or 5-10 membered heteroaryl;
L¹ is selected from bond, C₁₋₆ alkylene, C₁₋₆ haloalkylene, C₃₋₇ cycloalkylene, 4-7 membered heterocycloalkylene, 5-6 membered heteroarylene, —C₃₋₇ cycloalkylene-C₁₋₄ alkyl-, -(4-7 membered heterocycloalkylene)—C₁₋₄ alkyl-, -(5-6 membered heteroarylene)—C₁₋₄ alkyl-, —O—, —N(R^{L})—, —C(O)—, —C(O)N(R^{L})—, —N(R^{L})C(O)N(R^{L})—, —N(R^{L})C(O)O—, —S(O)₂—, —N(R^{L})S(O)₂—, and —N(R^{L})S(O)₂N(R^{L})—, wherein the C₁₋₆ alkylene, C₁₋₆ haloalkylene, C₃₋₇ cycloalkylene, 4-7 membered heterocycloalkylene, 5-6 membered heteroarylene, C₃₋₇ cycloalkylene-C₁₋₄ alkyl, (4-7 membered heterocycloalkylene)—C₁₋₄ alkyl, and (5-6 membered heteroarylene)—C₁₋₄ alkyl of L¹ are each optionally substituted with 1, 2, 3, or 4 independently selected R^{G} substituents;
L² is selected from bond, C₁₋₆ alkylene, C₁₋₆ haloalkylene, C₃₋₇ cycloalkylene, 4-7 membered heterocycloalkylene, 5-6 membered heteroarylene, —C₃₋₇ cycloalkylene-C₁₋₄ alkyl-, -(4-7 membered heterocycloalkylene)—C₁₋₄ alkyl-, -(5-6 membered heteroarylene)—C₁₋₄ alkyl-, —O—, —N(R^{L})—, —C(O)—, —C(O)N(R^{L})—, —N(R^{L})C(O)N(R^{L})—, —N(R^{L})C(O)O—, —S(O)₂—, —N(R^{L})S(O)₂—, and —N(R^{L})S(O)₂N(R^{L})—, wherein the C₁₋₆ alkylene, C₁₋₆ haloalkylene, C₃₋₇ cycloalkylene, 4-7 membered heterocycloalkylene, 5-6 membered heteroarylene, C₃₋₇ cycloalkylene-C₁₋₄ alkyl, (4-7 membered heterocycloalkylene)—C₁₋₄ alkyl, and (5-6 membered heteroarylene)—C₁₋₄ alkyl of L² are each optionally substituted with 1, 2, 3, or 4 independently selected R^{G} substituents;
each R^{L} is independently selected from H, C₁₋₆ alkyl, and C₁₋₆ haloalkyl;
R¹ is selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R¹ are each optionally substituted with 1, 2, 3, 4, 5, or 6 independently selected R^{G} substituents;
R² is selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a2}, —SR^{a2}—NR²R^{d2}, —C(O)R^{a2}, —C(O)OR^{a2}, —C(O)NR^{c2}R^{d2}, —C(O)NR^{c2}(OR^{a2}), —OC(O)NR²R^{d2}—NR^{c2}C(O)R^{a2}, —NR^{c2}C(O)OR^{a2}, —NR^{c2}C(O)NR^{c2}R^{d2}—NR²S(O)₂R^{b2}-R^{c2}S(O)₂NR^{c2}R^{d2}—NR^{c2}0R^{a2}, —NR^{c2}S(O)R^{b2}, —NR^{c2}S(O)NR^{c2}R^{d2}, —S(O)R^{b2}, —S(O)₂R^{b2}, —S(O)NR^{c2}R^{d2}, —S(O)₂NR^{c2}R^{d2}, —C(═NR^{c2})R^{a2}, —C(═NR^{c2})NR^{c2}R^{d2}—NR^{c2}C(═NR^{c2})R^{a2}—NR²C(═NR^{c2})NR^{c2}R^{d2}-R^{c2}S(O)(═NR^{c2})R^{b2}—NR^{c2}S(O)(═NR^{c2})NR²R^{d2}—S(O)(═NR^{c2})R^{b2}, and —S(O)(═NR^{c2})NR^{c2}R^{d2}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R² are each optionally substituted with 1, 2, 3, 4, 5, or 6 independently selected R^{G} substituents;
each R^{a2}, R^{b2}, R^{c2}, and R^{d2} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a2}, R^{b2}, R^{c2}, and R^{d2} are each optionally substituted with 1, 2, 3, 4, 5, or 6 independently selected R^{G} substituents;
or, any R^{c2} and R^{d2} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents; and
each R^{c2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
R³ is selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a3}, —SR^{a3}—NR^{c3}R^{d3}—C(O)R³, —C(O)OR^{a3}, —C(O)NR^{c3}R^{d3}, —C(O)NR^{c3}(OR^{a3}), —OC(O)NR^{c3}R^{d3}—NR^{c3}C(O)R^{a3}, —NR^{c3}C(O)OR^{a3}, —NR^{c3}C(O)NR^{c3}R^{d3}—NR^{c3}S(O)₂R^{b3}, —NR^{c3}S(O)₂NR³R^{d3}—NR^{c3}OR^{a3}, —NR^{c3}S(O)R^{b3}, —NR^{c3}S(O)NR^{c3}R^{d3}, —S(O)R^{b3}, —S(O)₂R¹³, —S(O)NR^{c3}R^{d3}, —S(O)₂NR^{c3}R^{d3}, —C(═NR^{c3})R^{a3}, —C(═NR^{c3})NR^{c3}R^{d3}—NR^{c3}C(═NR^{c3})R^{a3}, —NR^{c3}C(═NR^{c3})NR^{c3}R^{d3}, —NR³S(O)(═NR^{c3})R^{b3}, —NR^{c3}S(O)(═NR^{c3})NR³R^{d3}—S(O)(═NR^{c3})R^{b3}, and —S(O)(═NR^{c3})NR^{c3}R^{d3}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R³ are each optionally substituted with 1, 2, 3, 4, 5, or 6 independently selected R^{G} substituents;
each R^{a3}, R^{b3}, R^{c3}, and R^{d3} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a3}, R^{b3}, R^{c3}, and R^{d3} are each optionally substituted with 1, 2, 3, 4, 5, or 6 independently selected R^{G} substituents;
or, any R^{c3} and R^{d3} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents; and
each R^{c3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
R⁴ is selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a4}, —SR^{a4}—NR^{c4}R^{d4}—C(O)R^{a4}, —C(O)OR^{a4}, —C(O)NR^{c4}R^{d4}, —C(O)NR^{c4}(OR^{a4}), —OC(O)NR^{c4}R^{d4}—NR^{c4}C(O)R^{a4}, —NR^{c4}C(O)OR^{a4}, —NR^{c4}C(O)NR^{c4}R^{d4}, —NR^{c4}S(O)₂R^{b4}, —NR^{c4}S(O)₂NR^{c4}R^{d4}, —NR^{c4}OR^{a4}, —NR^{c4}S(O)R^{b4}, —NR^{c4}S(O)NR^{c4}R^{d4}, —S(O)R^{b4}, —S(O)₂R^{b4}, —S(O)NR^{c4}R^{d4}, —S(O)₂NR^{c4}R^{d4}, —C(═NR^{c4})R^{a4}, —C(═NR^{c4})NR^{c4}R^{d4}—NR^{c4}C(═NR^{c4})R^{a4}, —NR^{c4}C(═NR^{c4})NR^{c4}R^{d4}—NR^{c4}S(O)(═NR^{c4})R^{b4}, —NR^{c4}S(O)(═NR^{c4})NR^{c4}R^{d4}—S(O)(═NR^{c4})R^{b4}, and —S(O)(═NR^{c4})NR^{c4}R^{d4}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁴ are each optionally substituted with 1, 2, 3, 4, 5, or 6 independently selected R^{G} substituents;
each R^{a4}, R^{b4}, R^{c4}, and R^{d4} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a4}, R^{b4}, R^{c4}, and R^{d4} are each optionally substituted with 1, 2, 3, 4, 5, or 6 independently selected R^{G} substituents;
or, any R^{c4} and R^{d4} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents; and
each R^{c4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
R⁵ is selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁵ are each optionally substituted with 1, 2, 3, 4, 5, or 6 independently selected R^{G} substituents;
or, R⁴ and R⁵ taken together with the carbon atom to which they are attached form a C₃₋₇ cycloalkyl, or 4-7 membered heterocycloalkyl group, wherein the C₃₋₇ cycloalkyl, and 4-7 membered heterocycloalkyl are each optionally substituted with 1, 2, 3, or 4 independently selected R^{G} substituents;
R⁶ is selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, (5-6 membered heteroaryl)-C₁₋₄ alkyl, —CN, —OR^{a6}, —SR^{a6}—NR⁶R^{d6}, —C(O)R^{a6}, —C(O)OR^{a6}, —C(O)NR⁶R^{d6}, —C(O)NR^{c6}(OR^{a6}), —OC(O)NR⁶R^{d6}—NR^{c6}C(O)R^{a6}, —NR⁶C(O)OR^{a6}, —NR^{c6}C(O)NR⁶R^{d6}—NR⁶S(O)₂R^{b6}, —NR^{c6}S(O)₂NR^{c6}R^{d6}—NR^{o}60R^{a6}, —NR^{c6}S(O)R^{b6}, —NR^{c6}S(O)NR^{c6}R^{d6}, —S(O)R^{b6}, —S(O)₂R⁶, —S(O)NR^{c6}R^{d6}, —S(O)₂NR^{c6}R^{d6}, —C(═NR^{c6})R^{a6}, —C(═NR^{c6})NR^{c6}R^{d6}—NR^{c6}C(═NR^{c6})R^{a6}—NR⁶C(═NR^{c6})NR^{c6}R^{d6}-R^{c6}S(O)(═NR^{c6})R^{b6}, —NR^{c6}S(O)(═NR^{c6})NR⁶R^{d6}—S(O)(═NR^{c6})R^{b6}, and —S(O)(═NR^{c6})NR^{c6}R^{d6}, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R⁶ are each optionally substituted with 1, 2, 3, 4, 5, or 6 independently selected R^{G} substituents;
each R^{a6}, R^{b6}, R^{c6}, and R^{d6} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl of R^{a6}, R^{b6}, R^{c6}, and R^{d6} are each optionally substituted with 1, 2, 3, 4, 5, or 6 independently selected R^{G} substituents;
or, any R^{c6} and R^{d6} attached to the same N atom, together with the N atom to which they are attached, form a 4-7 membered heterocycloalkyl group, wherein the 4-7 membered heterocycloalkyl group is optionally substituted with 1, 2, 3, 4, 5, or 6 independently selected R^{G} substituents; and
each R^{c6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, (4-7 membered heterocycloalkyl)-C₁₋₄ alkyl, and (5-6 membered heteroaryl)-C₁₋₄ alkyl;
each R⁷ is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a7}, —SR^{a7}, —NR^{c7}R^{d7}, —NO₂, —C(O)R^{a7}, —C(O)OR^{a7}, —C(O)NR^{c7}R^{d7}, —C(O)NR^{c7}(OR^{a7}), —OC(O)R^{a7}, —OC(O)NR^{c7}R^{d7}, —OC(O)OR^{a7}, —OS(O)₂R^{b7}, —OS(O)₂NR^{c7}R^{d7}—NR^{c7}C(O)R^{a7}, —NR^{c7}C(O)OR^{a7}, —NR⁷C(O)NR^{c7}R^{d7}—NR^{c7}S(O)₂R^{b7}—NR^{c7}S(O)₂NR^{c7}R^{d7}—NR^{o}70R^{a7}, —NR^{c7}S(O)R^{b7}, —NR^{c7}S(O)NR^{c7}R^{d7}, —S(O)R^{b7}, —S(O)₂R^{b7}, —S(O)NR^{c7}R^{d7}, —S(O)₂NR^{c7}R^{d7}, —C(═NR^{c7})R^{a7}, —C(═NR^{c7})NR^{c7}R^{d7}—NR^{c7}C(═NR^{c7})R^{a7}, —NR⁷C(═NR^{c7})NR^{c7}R^{d7}-R^{c7}S(O)(═NR^{c7})R^{b7}, —NR^{c7}S(O)(═NR^{c7})NR^{c7}R^{d7}—OS(O)(═NR^{c7})R^{b7}, —S(O)(═NR^{c7})R^{b7}, —S(O)(═NR^{c7})NR^{c7}R^{d7}, —C(O)NR^{c7}S(O)₂R^{b7}, —C(O)NR^{c7}S(O)₂NR^{c7}R^{d7}—S(O)₂NR^{c7}C(O)R^{b7}, —NR^{c7}S(O)NR^{c7}C(O)R^{b7}, and —P(O)R⁷R^{g7}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁷ are each optionally substituted with 1, 2, 3, 4, 5, or 6 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a7}, R^{c7}, and R^{d7} are each optionally substituted with 1, 2, 3, 4, 5, or 6 independently selected R^{7A} substituents;
or, any R^{c7} and R^{d7} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C6.10 aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b7} are each optionally substituted with 1, 2, 3, 4, 5, or 6 independently selected R^{7A} substituents;
each R^{c7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f7} and R^{g7} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{7A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a7A}, —SR^{a7A}, —N^{c7A}, R^{d7A}, —NO₂, —C(O)R^{a7A}, —C(O)OR^{a7A}, —C(O)NR^{c7A}, R^{d7A}, —C(O)NR^{c7A}(OR^{a7A}), —OC(O)R^{a7A}, —OC(O)NR^{c7A}, R^{d7A}, —OC(O)OR^{a7A}, —OS(O)₂R^{b7A}, OS(O)₂NR^{7A}, R^{d7A}, —NR^{c7a}C(O)R^{a7A}, —NR^{c7a}C(O)OR^{a7A}, —NR^{c7a}C(O)NR^{c7A}, R^{d7A}, —NR^{c7}AS(O)₂R^{b7A} NR^{c7}AS(O)₂NR^{c7A}, R^{d7A}, —NR^{c7A}OR^{a7A}, —NR^{c7}AS(O)R^{b7A}, —NR^{c7}AS(O)NR^{c7A}, R^{d7A}, —S(O)R^{b7A}, —S(O)₂R^{b7A}, —S(O)NR^{c7A}, R^{d7A}, —S(O)₂NR^{c7A}, R^{d7A}, —C(═NR^{e7A})R^{a7A}, —C(═NR^{c7A})R^{c7A}, R^{d7A}, —NR^{c7a}C(═NR^{c7A})R^{a7A}, —NR^{c7a}C(═NR^{e7A})NR^{c7A}, R^{d7A} N^{c7}AS(O)(═NR^{c7A})R^{b7A}, —NR^{c7}AS(O)(═NR^{c7A})NR^{c7A}, R^{d7A}, —OS(O)(═NR^{c7A})R^{b7A}, —S(O)(═NR^{c7A})R^{b7A}, —S(O)(═NR^{c7A})R^{c7A}, R^{d7A}, —C(O)NR^{c7}AS(O)₂R^{b7A}, —C(O)NR^{c7}AS(O)₂NR^{c7A}, R^{d7A}, —S(O)₂NR^{c7a}C(O)R^{b7A}—NR^{c7}AS(O)NR^{c7a}C(O)R^{b7A}, and —P(O)R^{f7A}R^{g7A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{7A} are each optionally substituted with 1, 2, 3, 4, 5, or 6 independently selected R^{G} substituents;
each R^{a7A}, R^{c7A}, and R^{d7A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a7A}, R^{c7}A and R^{d7A} are each optionally substituted with 1, 2, 3, 4, 5, or 6 independently selected R^{G} substituents;
or, any R^{c7A} and R^{d7A} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{b7A} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b7A} are each optionally substituted with 1, 2, 3, 4, 5, or 6 independently selected R^{G} substituents;
each R^{e7A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f7A} and R^{g7A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f7A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a8}, —SR^{a8}, —NR^{c8}R^{d8}, —NO₂, —C(O)R^{a8}, —C(O)OR^{a8}, —C(O)NR^{c8}R^{d8}—C(O)NR^{c8}(OR^{a8}), —OC(O)R^{a8}, —OC(O)NR^{c8}R^{d8}, —OC(O)OR^{a8}, —OS(O)₂R^{b8}, —OS(O)₂NR^{c8}R^{d8}—NR^{c8}C(O)R^{a8}, —NR^{c8}C(O)OR^{a8}, —NR^{c8}C(O)NR^{c8}R^{d8}—NR^{c8}S(O)₂R^{b8}, —NR^{c8}S(O)₂NR^{c8}R^{d8}—NR^{c8}OR^{a8}, —NRS(O)RV, —NR^{c8}S(O)NR⁸R^{d8}, —S(O)R^{b8}, —S(O)₂R^{b8}, —S(O)NR^{c8}R^{d8}, —S(O)₂NR^{c8}R^{d8}, —C(═NR^{c8})R^{a8}, —C(═NR^{c8})NR^{c8}R^{d8}—NR^{c8}C(═NR^{c8})R^{a8}, —NR^{c8}C(═NR^{c8})NR^{c8}R^{d8}—NR^{c8}S(O)(═NR^{c8})RV, —NR^{c8}S(O)(NR^{e8})NR^{c8}R^{d8}—OS(O)(═NR^{c8})R^{b8}, —S(O)(═NR^{c8})R^{b8}, —S(O)(═NR^{c8}R)N ⁸R^{d8}, —C(O)NR^{c8}S(O)₂Rb, —C(O)NR^{c8}S(O)₂NR^{c8}R^{d8}—S(O)₂NR^{c8}C(O)R^{b8}, —NR^{c8}S(O)NR^{c8}C(O)RV, and —P(O)R^{b8}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{f7A} are each optionally substituted with 1, 2, 3, 4, 5, or 6 independently selected R^{8A} substituents;
each R^{a8}, R^{c8}, and R^{d8} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{a8}, R^{c8}, and R^{d8} are each optionally substituted with 1, 2, 3, 4, 5, or 6 independently selected R^{8A} substituents;
or, any R^{b8} and R^{d8} attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{8A} substituents;
each R^{b8} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b8} are each optionally substituted with 1, 2, 3, 4, 5, or 6 independently selected R^{8A} substituents;
each R⁸ is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R and R⁹⁸ are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{8A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a8A}, —SR^{a8A}, —NR^{8A}R^{d8A}, —NO₂, —C(O)R^{a8A}, —C(O)OR^{a8A}, —C(O)NR^{c8A}, R^{d8A}, —C(O)NR^{c8A}(OR^{a8A}), —OC(O)R^{a8A}, —OC(O)NR^{c8A}, R^{d8A}, —OC(O)OR^{a8A}, —OS(O)₂R^{b8A}, —OS(O)₂NR^{8A}, R^{d8A}, —NR^{c8a}C(O)R^{a8A}, —NR^{c8a}C(O)OR^{a8A}, —NR^{8a}C(O)NR^{c8A}, R^{d8A}, —NR⁸AS(O)₂R^{b8A}, —NR^{c8}AS(O)₂NR^{c8A}, R^{d8A}, —NR^{c8A}OR^{a8A}, —NR^{c8}AS(O)R^{b8A}, —NR⁸AS(O)NR^{c8A}, R^{d8A}, —S(O)R^{b8A}, —S(O)₂R^{b8A}, —S(O)NR^{c8A}, R^{d8A}, —S(O)₂NR^{c8A}, R^{d8A}, —C(═NR^{c8A})R^{a8A}, -C(═NR^{e8A})R^{8A}, R^{d8A}, —NR^{c8a}C(═NR^{c8A})R^{a8A}, —NR^{c8a}C(═NR^{e8A})NR^{8A}, R^{d8A}, —R⁸AS(O)(═NR^{c8A})R^{b8A}, —NR^{c8}AS(O)(═NR^{c8A})NR^{8A}, R^{d8A}, —OS(O)(═NR^{c8A})R^{b8A}, —S(O)(═NR^{c8A})R^{b8A}, —S(O)(═NR^{c8A})R^{8A}, R^{d8A}, —C(O)NR^{c8}AS(O)₂R^{b8A}, —C(O)NR^{c8}AS(O)₂NR^{c8A}, R^{d8A}, —S(O)₂NR^{c8a}C(O)R^{b8A}, —NR^{c8}AS(O)NR^{c8a}C(O)R^{b8A}, and —P(O)R^{g8A}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{8A} are each optionally substituted with 1, 2, 3, 4, 5, or 6 independently selected R^{G} substituents;
each R^{a8A}, R^{b8A}, and R^{d8A}, is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆0.1o aryl-C₁₋₄ alkyl, (4-10 membered heterocycloalkyl)-C₁₋₄ alkyl, and (5-10 membered heteroaryl)-C₁₋₄ alkyl of R^{a8A}, R^{c8}A and R^{d8A}, are each optionally substituted with 1, 2, 3, 4, 5, or 6 independently selected R^{G} substituents;
or, any R^{b8A} and R^{d8A}, attached to the same N atom, together with the N atom to which they are attached, form a 4-10 membered heterocycloalkyl group, wherein the 4-10 membered heterocycloalkyl group is optionally substituted with 1, 2, 3, 4, 5, or 6 independently selected R^{G} substituents;
each R^{b8A} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl, wherein the C₁₋₆ alkyl, C₁₋₆ haloalkyl, C₂₋₆ alkenyl, C₂₋₆ alkynyl, C₃₋₁₀ cycloalkyl, C6.10 aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R^{b8A} are each optionally substituted with 1, 2, 3, 4, 5, or 6 independently selected R^{G} substituents;
each R^{c8A} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{f8A} and R^{g8A} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{G} is independently selected from OH, CN, halo, oxo, C₁₋₄ alkyl, C₂₋₄ alkenyl, C₂₋₄ alkynyl, C₁₋₄ haloalkyl, cyano-C₁₋₄ alkyl, HO—C₁₋₄ alkyl, C₁₋₄ alkoxy-C₁₋₄ alkyl, C₃₋₇ cycloalkyl, 4-7 membered heterocycloalkyl, C₁₋₄ alkoxy, C₁₋₄ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, aminocarbonyloxy, C₁₋₃ alkylaminocarbonyloxy, di(C₁₋₃ alkyl)aminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

2. The compound of claim 1, or a pharmaceutically acceptable salt thereof, wherein X¹ is NR¹.

3. The compound of claim 1 or 2, or a pharmaceutically acceptable salt thereof, wherein R¹ is selected from H, C₁₋₆ alkyl, C₂₋₆ alkenyl, C₂₋₆ alkynyl, and C₁₋₆ haloalkyl.

4. The compound of claim 1 or 2, or a pharmaceutically acceptable salt thereof, wherein R¹ is C₁₋₆ alkyl.

5. The compound of claim 1 or 2, or a pharmaceutically acceptable salt thereof, wherein R¹ is methyl.

6. The compound of any one of claims 1 to 5, or a pharmaceutically acceptable salt thereof, wherein X² is CR².

7. The compound of any one of claims 1 to 6, or a pharmaceutically acceptable salt thereof, wherein R² is selected from H, C₁₋₆ alkyl, C₂₋₆ alkenyl, C₂₋₆ alkynyl, and C₁₋₆ haloalkyl.

8. The compound of any one of claims 1 to 6, or a pharmaceutically acceptable salt thereof, wherein R² is H.

9. The compound of any one of claims 1 to 8, or a pharmaceutically acceptable salt thereof, wherein X³ is CR³.

10. The compound of any one of claims 1 to 9, or a pharmaceutically acceptable salt thereof, wherein R³ is selected from H, C₁₋₆ alkyl, C₂₋₆ alkenyl, C₂₋₆ alkynyl, and C₁₋₆ haloalkyl.

11. The compound of any one of claims 1 to 9, or a pharmaceutically acceptable salt thereof, wherein R³ is H.

12. The compound of any one of claims 1 to 11, or a pharmaceutically acceptable salt thereof, wherein R⁴ is selected from H, halo, C₁₋₆ alkyl, C₂₋₆ alkenyl, C₂₋₆ alkynyl, and C₁₋₆ haloalkyl.

13. The compound of any one of claims 1 to 11, or a pharmaceutically acceptable salt thereof, wherein R⁴ is selected from H and C₁₋₆ alkyl.

14. The compound of any one of claims 1 to 11, or a pharmaceutically acceptable salt thereof, wherein R⁴ is H.

15. The compound of any one of claims 1 to 14, or a pharmaceutically acceptable salt thereof, wherein R⁵ is selected from H, halo, C₁₋₆ alkyl, C₂₋₆ alkenyl, C₂₋₆ alkynyl, and C₁₋₆ haloalkyl.

16. The compound of any one of claims 1 to 14, or a pharmaceutically acceptable salt thereof, wherein R⁵ is selected from H and C₁₋₆ alkyl.

17. The compound of any one of claims 1 to 14, or a pharmaceutically acceptable salt thereof, wherein R⁵ is H.

18. The compound of any one of claims 1 to 17, or a pharmaceutically acceptable salt thereof, wherein R⁶ is selected from H, halo, C₁₋₆ alkyl, C₂₋₆ alkenyl, C₂₋₆ alkynyl, and C₁₋₆ haloalkyl.

19. The compound of any one of claims 1 to 17, or a pharmaceutically acceptable salt thereof, wherein R⁶ is selected from H and C₁₋₆ alkyl.

20. The compound of any one of claims 1 to 17, or a pharmaceutically acceptable salt thereof, wherein R⁶ is methyl.

21. The compound of any one of claims 1 to 20, or a pharmaceutically acceptable salt thereof, wherein Ring A is C₃₋₁₄ cycloalkyl.

22. The compound of any one of claims 1 to 20, or a pharmaceutically acceptable salt thereof, wherein Ring A is a bicyclic C₇₋₁₄ cycloalkyl.

23. The compound of any one of claims 1 to 20, or a pharmaceutically acceptable salt thereof, wherein Ring A is dihydroindenyl.

24. The compound of any one of claims 1 to 23, or a pharmaceutically acceptable salt thereof, wherein m is 0.

25. The compound of any one of claims 1 to 20, or a pharmaceutically acceptable salt thereof, wherein Ring A is

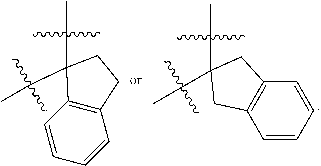 [SMILES: *C1(*)CCc2ccccc21 ; *C1(*)Cc2ccccc2C1]

26. The compound of any one of claims 1 to 25, or a pharmaceutically acceptable salt thereof, wherein L¹ is selected from bond, C₁₋₆ alkylene, and C₁₋₆ haloalkylene.

27. The compound of any one of claims 1 to 25, or a pharmaceutically acceptable salt thereof, wherein L¹ is C₁₋₆ alkylene.

28. The compound of any one of claims 1 to 25, or a pharmaceutically acceptable salt thereof, wherein L¹ is —CH(CH₃)-.

29. The compound of any one of claims 1 to 28, or a pharmaceutically acceptable salt thereof, wherein L² is selected from —N(R^{L})—, —C(O)—, and —C(O)N(R^{L})-.

30. The compound of any one of claims 1 to 28, or a pharmaceutically acceptable salt thereof, wherein L² is —N(R^{L})—.

31. The compound of any one of claims 1 to 30, or a pharmaceutically acceptable salt thereof, wherein each R^{L} is independently selected from H and C₁₋₆ alkyl.

32. The compound of any one of claims 1 to 28, or a pharmaceutically acceptable salt thereof, wherein L² is —NH—.

33. The compound of any one of claims 1 to 32, or a pharmaceutically acceptable salt thereof, wherein Ring B is C₆₋₁₀ aryl or 5-10 membered heteroaryl.

34. The compound of any one of claims 1 to 32, or a pharmaceutically acceptable salt thereof, wherein Ring B is phenyl or 5-6 membered heteroaryl.

35. The compound of any one of claims 1 to 32, or a pharmaceutically acceptable salt thereof, wherein Ring B is phenyl or pyridinyl.

36. The compound of any one of claims 1 to 35, or a pharmaceutically acceptable salt thereof, wherein n is 1 or 2.

37. The compound of any one of claims 1 to 36, or a pharmaceutically acceptable salt thereof, wherein each R⁷ is independently selected from halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, CN, —OR⁷, —NR^{c7}R^{d7}, —C(O)R^{a7}, —C(O)OR^{a7}, and —C(O)NR^{c7}R^{d7}, wherein the C₁₋₆ alkyl, C₂₋₆ alkenyl, C₂₋₆ alkynyl, C₃₋₁₀ cycloalkyl, C₆₋₁₀ aryl, 4-10 membered heterocycloalkyl, and 5-10 membered heteroaryl are each optionally substituted with 1, 2, 3, or 4 independently selected R^{7A} substituents; and
each R^{a7}, R^{c7}, and R^{d7} is independently selected from H, C₁₋₆ alkyl and C₁₋₆ haloalkyl.

38. The compound of any one of claims 1 to 36, or a pharmaceutically acceptable salt thereof, wherein each R⁷ is independently selected from halo, C₁₋₆ alkyl, C₁₋₆ haloalkyl, C₃₋₁₀ cycloalkyl, C₆₋₁₀ aryl, 4-10 membered heterocycloalkyl, 5-10 membered heteroaryl, —C(O)R^{a7}, —C(O)OR^{a7}, and —C(O)NR^{c7}R^{d7}, wherein the C₁₋₆ alkyl, C₁₋₆ haloalkyl, C₃₋₁₀ cycloalkyl, C₆₋₁₀ aryl, 4-10 membered heterocycloalkyl, and 5-10 membered heteroaryl are each optionally substituted with 1, 2, 3, or 4 independently selected R^{7A} substituents; and
each R^{a7}, R^{c7}, and R^{d7} is independently selected from H, C₁₋₆ alkyl and C₁₋₆ haloalkyl.

39. The compound of any one of claims 1 to 36, or a pharmaceutically acceptable salt thereof, wherein each R⁷ is independently selected from halo, C₁₋₆ alkyl, 5-6 membered heteroaryl, and —C(O)OR^{a7}, wherein the C₁₋₆ alkyl and 5-6 membered heteroaryl is optionally substituted with 1, 2, 3, or 4 independently selected R^{7A} substituents; and
each R^{a7} is independently selected from H and C₁₋₆ alkyl.

40. The compound of any one of claims 1 to 39, or a pharmaceutically acceptable salt thereof, wherein each R^{7A} is independently selected from oxo, halo, C₁₋₆ alkyl, C₁₋₆ haloalkyl, C₂₋₆ alkenyl, and C₂₋₆ alkynyl.

41. The compound of any one of claims 1 to 39, or a pharmaceutically acceptable salt thereof, wherein each R^{7A} is independently selected from halo and C₁₋₆ alkyl.

42. The compound of any one of claims 1 to 36, or a pharmaceutically acceptable salt thereof, wherein each R⁷ is independently selected from chloro, methyl, COOH, fluoropyridinyl, and methylpyrazolyl.

43. The compound of claim 1, or a pharmaceutically acceptable salt thereof, wherein:
X¹ is NR¹ or O;
X² is CR² or N;
X³ is CR³ or N;
n is 0, 1, 2, 3, or 4;
m is 0, 1, 2, 3, or 4;
Ring A is C₃₋₁₄ cycloalkyl or 4-14 membered heterocycloalkyl;
Ring B is C₃₋₁₀ cycloalkyl, C₆₋₁₀ aryl, 4-10 membered heterocycloalkyl, or 5-10 membered heteroaryl;
L¹ is selected from bond, C₁₋₆ alkylene, and C₁₋₆ haloalkylene;
L² is selected from —N(R^{L})—, —C(O)—, and —C(O)N(R^{L})—;
each R^{L} is independently selected from H, C₁₋₆ alkyl, and C₁₋₆ haloalkyl;
R¹ is selected from H, C₁₋₆ alkyl, C₂₋₆ alkenyl, C₂₋₆ alkynyl, and C₁₋₆ haloalkyl;
R² is selected from H, C₁₋₆ alkyl, C₂₋₆ alkenyl, C₂₋₆ alkynyl, and C₁₋₆ haloalkyl;
R³ is selected from H, C₁₋₆ alkyl, C₂₋₆ alkenyl, C₂₋₆ alkynyl, and C₁₋₆ haloalkyl;
R⁴ is selected from H, halo, C₁₋₆ alkyl, C₂₋₆ alkenyl, C₂₋₆ alkynyl, and C₁₋₆ haloalkyl;
R⁵ is selected from H, halo, C₁₋₆ alkyl, C₂₋₆ alkenyl, C₂₋₆ alkynyl, and C₁₋₆ haloalkyl;
R⁶ is selected from H, halo, C₁₋₆ alkyl, C₂₋₆ alkenyl, C₂₋₆ alkynyl, and C₁₋₆ haloalkyl;
each R⁷ is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a7}, —SR^{a7}, —NR^{c7}R^{d7}, —NO₂, —C(O)R^{a7}, —C(O)OR^{a7}, —C(O)NR^{c7}R^{d7}, —C(O)NR^{c7}(OR^{a7}), —OC(O)R^{a7}, —OC(O)NR^{c7}R^{d7}, —OC(O)OR^{a7}, —OS(O)₂R^{b7}, —OS(O)₂NR^{c7}R^{d7}—NR^{c7}C(O)R^{a7}—NR^{c7}C(O)OR^{a7}, —NR⁷C(O)NR^{c7}R^{d7}—NR⁷S(O)₂R^{b7}—NR^{c7}S(O)₂NR^{c7}R^{d7}—NR^{o}70R^{a7}, —NR^{c7}S(O)R^{b7}, —NR^{c7}S(O)NR^{c7}R^{d7}, —S(O)R⁷, —S(O)₂R^{b7}, —S(O)NR^{c7}R^{d7}, and —S(O)₂NR^{c7}R^{d7}, wherein the C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl of R⁷ are each optionally substituted with 1, 2, 3, 4, 5, or 6 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
or, any R^{c7} and R^{d7} attached to the same N atom, together with the N atom to which they are attached, form a 4-10 membered heterocycloalkyl group;
each R^{b7} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{7A} is independently selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, —CN, —OR^{a7A}, —SR^{a7A}, —N^{c7A}, R^{d7A}, —NO₂, —C(O)R^{a7A}, —C(O)OR^{a7A}, —C(O)NR^{c7A}, R^{d7A}, —C(O)NR^{c7A}(OR^{a7A}), —OC(O)R^{a7A}, —OC(O)NR^{c7A}, R^{d7A}, —OC(O)OR^{a7A}, —OS(O)₂R^{b7A}, —OS(O)₂NR^{7A}, R^{d7A}, —NR^{c7a}C(O)R^{a7A}, —NR^{c7a}C(O)OR^{a7A}, —NR^{c7a}C(O)NR^{c7A}, R^{d7A}, —NR^{c7}AS(O)₂R^{b7A}, —R^{c7}AS(O)₂NR^{c7A}, R^{d7A}, —NR^{c7A}OR^{a7A}, —NR^{c7}AS(O)R^{b7A}, —NR^{c7}AS(O)NR^{c7A}, R^{d7A}, —S(O)R^{b7A}, and —S(O)₂R^{b7}A;
each R^{a7A}, R^{c7A}, and R^{d7A} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
or, any R^{c7A} and R^{d7A} attached to the same N atom, together with the N atom to which they are attached, form a 4-10 membered heterocycloalkyl group;
each R^{b7A} is independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl; and
each R^{f7A} is independently selected from selected from oxo, halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, (5-10 membered heteroaryl)-C₁₋₄ alkyl, and —CN.

44. The compound of claim 1, or a pharmaceutically acceptable salt thereof, wherein:
X¹ is NR¹ or O;
X² is CR² or N;
X³ is CR³ or N;
n is 0, 1, 2, 3, or 4;
m is 0, 1, 2, 3, or 4;
Ring A is C₃₋₁₄ cycloalkyl or 4-14 membered heterocycloalkyl;
Ring B is C₃₋₁₀ cycloalkyl, C6₁o aryl, 4-10 membered heterocycloalkyl, or 5-10 membered heteroaryl;
L¹ is selected from bond, C₁₋₆ alkylene, and C₁₋₆ haloalkylene;
L² is selected from —N(R^{L})—, —C(O)—, and —C(O)N(R^{L})—;
each R^{L} is independently selected from H, C₁₋₆ alkyl, and C₁₋₆ haloalkyl;
R¹ is selected from H, C₁₋₆ alkyl, C₂₋₆ alkenyl, C₂₋₆ alkynyl, and C₁₋₆ haloalkyl;
R² is selected from H, C₁₋₆ alkyl, C₂₋₆ alkenyl, C₂₋₆ alkynyl, and C₁₋₆ haloalkyl;
R³ is selected from H, C₁₋₆ alkyl, C₂₋₆ alkenyl, C₂₋₆ alkynyl, and C₁₋₆ haloalkyl;
R⁴ is selected from H, halo, C₁₋₆ alkyl, C₂₋₆ alkenyl, C₂₋₆ alkynyl, and C₁₋₆ haloalkyl;
R⁵ is selected from H, halo, C₁₋₆ alkyl, C₂₋₆ alkenyl, C₂₋₆ alkynyl, and C₁₋₆ haloalkyl;
R⁶ is selected from H, halo, C₁₋₆ alkyl, C₂₋₆ alkenyl, C₂₋₆ alkynyl, and C₁₋₆ haloalkyl;
each R⁷ is independently selected from halo, C₁₋₆ alkyl, C₂₋₆ alkenyl, C₂₋₆ alkynyl, C₁₋₆ haloalkyl, C₃₋₁₀ cycloalkyl, C₆₋₁₀ aryl, 4-10 membered heterocycloalkyl, 5-10 membered heteroaryl, CN, —OR^{a7}, —NR^{c7}R^{d7}, —C(O)R^{a7}, —C(O)OR^{a7}, and —C(O)NR^{c7}R^{d7}, wherein the C₁₋₆ alkyl, C₂₋₆ alkenyl, C₂₋₆ alkynyl, C₃₋₁₀ cycloalkyl, C6.10 aryl, 4-10 membered heterocycloalkyl, and 5-10 membered heteroaryl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 4-10 membered heterocycloalkyl, 5-10 membered heteroaryl, C₃₋₁₀ cycloalkyl-C₁₋₄ alkyl, C₆₋₁₀ aryl-C₁₋₄ alkyl, (4-10 membered heterocycloalkyl)-C₁₋₄ alkyl, and (5-10 membered heteroaryl)-C₁₋₄ alkyl;
each R^{7A} is independently selected from oxo, halo, C₁₋₆ alkyl, C₁₋₆ haloalkyl, C₂₋₆ alkenyl, and C₂₋₆ alkynyl; and
each R^{f7A} is independently selected from selected from oxo, halo, C₁₋₆ alkyl, C₂₋₆ alkenyl, C₂₋₆ alkynyl, and C₁₋₆ haloalkyl.

45. The compound of claim 1, wherein the compound of Formula I is a compound of Formula II: 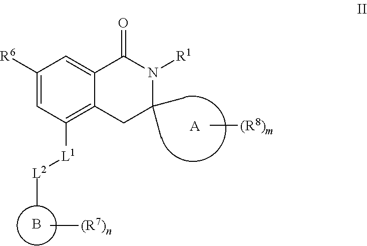
or a pharmaceutically acceptable salt thereof.

46. The compound of claim 1, wherein the compound of Formula I is a compound of Formula III: 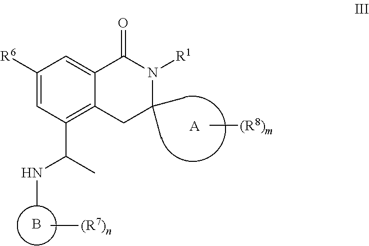
or a pharmaceutically acceptable salt thereof.

47. The compound of claim 1, which is selected from:
2-[1-(2,7-dimethyl-1-oxo-spiro[4H-isoquinoline-3,1′-indane]-5yl)ethylamino]benzoic acid;
3-[1-(2,7-dimethyl-1-oxo-spiro[4H-isoquinoline-3,1′-indane]-5-yl)ethylamino]-6-methyl-pyridine-2-carboxylic acid;
6-chloro-3-[1-(2,7-dimethyl-1-oxo-spiro[4H-isoquinoline-3,1′-indane]-5-yl)ethylamino]pyridine-2-carboxylic acid;
2-[1-(2,7-dimethyl-1-oxo-spiro[4H-isoquinoline-3,2′-indane]-5-yl)ethylamino]benzoic acid;
3-((1-(2′,7′-dimethyl-1′-oxo-1,1′,3,4′-tetrahydro-2′H-spiro[indene-2,3′-isoquinolin]-5′-yl)ethyl)amino)-6-methylpicolinic acid;
5-[1-[[6-chloro-2-(1-methylpyrazol-4-yl)-3-pyridyl]amino]ethyl]-2,7-dimethyl-spiro[4H-isoquinoline-3,1′-indane]-1-one; and
5-[1-[[6-chloro-2-(3-fluoro-4-pyridyl)-3-pyridyl]amino]ethyl]-2,7-dimethyl-spiro[4H-isoquinoline-3,1′-indane]-1-one;
or a pharmaceutically acceptable salt thereof.

48. A pharmaceutical composition, comprising a compound of any one of claims 1 to 47, or a pharmaceutically acceptable salt thereof, and a pharmaceutically acceptable carrier.

49. A method of inhibiting an activity of PI3Kα kinase, comprising contacting the kinase with a compound of any one of claims 1 to 47, or a pharmaceutically acceptable salt thereof.

50. A method of treating a PI3Kα-mediated disease or disorder in a patient, comprising administering to the patient a therapeutically effective amount of a compound of any one of claims 1 to 47, or a pharmaceutically acceptable salt thereof.

51. The method of claim 50, wherein the disease or disorder is a cancer.

52. The method of claim 51, wherein the cancer is selected from breast cancer, brain cancer, prostate cancer, endometrial cancer, gastric cancer, leukemia, lymphoma, sarcoma, colorectal cancer, lung cancer, ovarian cancer, skin cancer, and head and neck cancer.

53. The method of claim 50, wherein the disease or disorder is CLOVES syndrome (congenital lipomatous overgrowth, vascular malformations, epidermal naevi, scoliosis/skeletal and spinal syndrome), or PIK3CA-related overgrowth syndrome (PROS).
